(19) United States
(12) Patent Application Publication
(10) Pub. No.: US 2004/0180959 A1
(43) Pub. Date: Sep. 16, 2004

(54) Gabapentin analogues for fibromy algia and concomitant disorders

(76) Inventors: David James Dooley, South Lyon, MI (US); Charles Price Taylor JR., Chelsea, MI (US); Andrew John Thorpe, Whitmore Lake, MI (US); David Juergen Wustrow, Ann Arbor, MI (US)

Correspondence Address: WARNER-LAMBERT COMPANY, 2800 PLYMOUTH RD, ANN ARBOR, MI, 48105, US

(21) Appl. No.: 10/735,561

(22) Filed: Dec. 12, 2003

Related U.S. Application Data
(60) Provisional application No. 60/433,491, filed on Dec. 13, 2002.
(60) Provisional application No. 60/483,435, filed on Jun. 27, 2003.

(51) Int. Cl. A61K 31/195

(52) U.S. Cl. 514/561

(57) ABSTRACT

This invention provides new uses for compounds of formula

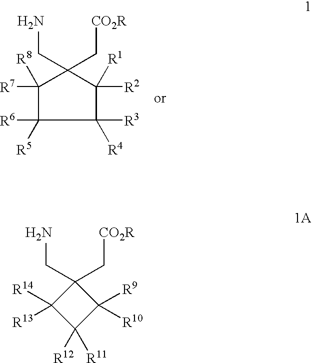

or a pharmaceutically acceptable salt thereof for the treatment of fibromyalgia and other disorders.

This application claims the benefit of U.S. Provisional Application Serial No. 60/433,491, filed Dec. 13, 2002, and U.S. Provisional Application Serial No. 60/483,435 filed Jun. 27, 2003; the entire contents of which applications are hereby incorporated herein by reference.

### BACKGROUND OF THE INVENTION

This invention relates to the use of certain alpha2delta ligands for the treatment of fibromyalgia and other central nervous system disorders. Fibromyalgia (FM) is a chronic syndrome characterized mainly by widespread pain, unrefreshing sleep, disturbed mood, and fatigue. The main symptoms fibromyalgia include pain, sleep, mood disturbances and fatigue. Syndromes commonly associated with fibromyalgia include irritable bowel syndrome, and migraine headaches, among others. Success of treating fibromyalgia with a single pharmacological agent has been characterized as modest and results of clinical trials have been characterized as disappointing. It is believed that based on current understanding of the mechanisms and pathways involved in fibromyalgia, multiple agents will be required, aimed at the major symptoms of pain, disturbed sleep, mood disturbances, and fatigue. Fibromyalgia patients are often sensitive to side effects of medications, a characteristic perhaps related to the pathophysiology of this disorder (Barkhuizen A, Rational and Targeted pharmacologic treatment of fibromyalgia. Rheum Dis Clin N Am 2002; 28: 261-290; Leventhal L J. Management of fibromyalgia. Ann Intern Med 1999;131:850-8).

While fibromyalgia is a complex disorder with multiple facets, this complexity can be well assessed (Yunus M B, A comprehensive medical evaluation of patients with fibromyalgia syndrome, Rheum Dis N Am 2002; 28:201-217). The diagnosis of FM is usually based on the 1990 recommendations of the American College of Rheumatology classification criteria (Bennett R M, The rational management of fibromyalgia patients. Rheum Dis Clin N Am 2002; 28: 181-199; Wolfe F, Smythe H A, Yunus M B, Bennett R M, Bombardier C, Goldenberg D L, et al. The American College of Rheumatology 1990 criteria for the classification of fibromyalgia: Report of the Multicenter Criteria Committee. Arthritis Rheum 1990; 33:160-72). Evaluation, management, and pharmacological treatment of fibromyalgia have been reviewed (Barkhuizen A, Rational and Targeted pharmacologic treatment of fibromyalgia. Rheum Dis Clin N Am 2002; Buskila D, Fibomyalgia, chronic fatigue syndrome and myofacial pain syndrome. Current opinions in Rheumatology 2001; 13: 117-127; Leventhal L J. Management of fibromyalgia. Ann Intern Med 1999;131:850-8; Bennett R M, The rational management of fibromyalgia patients. Rheum Dis Clin N Am 2002; 28: 181-199; Yunus M B, A comprehensive medical evaluation of patients with fibromyalgia syndrome, Rheum Dis N Am 2002; 28:201-217).

Gabapentin, pregabalin and other alpha2delta ligands including 4H-[1,2,4]oxadiazol-5-one, C-[1-(1H-Tetrazol-5-ylmethyl)-cycloheptyl]-methylamine, (3S,4S)-(1-Aminomethyl-3,4-dimethyl-cyclopentyl)-acetic acid, (1α,3α,5α)(3-amino-methyl-bicyclo[3.2.0]hept-3-yl)-acetic acid, and (3S ,5R)-3-aminomethyl-5-methyl-heptanoic acid, and pharmaceutically acceptable salts and solvates thereof, are referred to in U.S. Pat. No. 4,024,175; U.S. Pat. Nos. 4,087,544; 6,306,910; WO9921824, WO0190052, WO0128978, EP0641330, WO9817627, and WO0076958,. The foregoing patents and applications are incorporated herein by reference in their entirety.

U.S. patent application Ser. No. 09/485,382 filed Feb. 8, 2000, refers to compounds of formulas 1 and 1A below. Application Ser. No. 09/485382 and U.S. patent application Ser. No. 10/297,827, filed May 18, 2001 disclose various utilities for the compounds of formula 1 and 1A below. The entire contents of applications Ser. Nos. 09/485,382 and 10/297,827 are hereby incorporated herein by reference.

### SUMMARY OF THE INVENTION

This invention relates to a method for treating a disorder in a mammal, including a human, comprising administering to said mammal a therapeutically effective amount of a compound of formula 1 or 1A

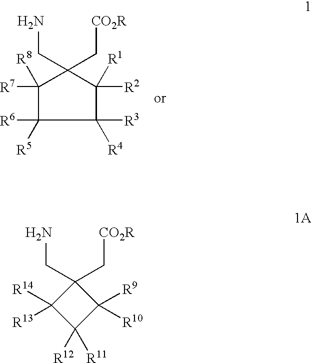

or a pharmaceutically acceptable salt thereof wherein:

R is hydrogen or a lower alkyl;

R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen., and wherein said disorder is selected from obsessive-compulsive disorder (OCD), phobias, post traumatic stress disorder (PTSD), and fibromyalgia.

A more specific embodiment of this invention relates to the above method wherein the disorder being treated is a phobia selected from agoraphobia, agoraphobia without history of panic disorder, specific phobia, and social phobia.

Another more specific embodiment of this invention relates to the above method wherein the compound administered is (3S, 4S)-(1-Aminomethyl-3,4-dimethyl-cyclopentyl)-acetic acid or a pharmaceutically acceptable salt thereof.

Another more specific embodiment of this invention relates to the above method wherein the compound administered is (3S, 4S)-(1-Aminomethyl-3,4-dimethyl-cyclopentyl)-acetic acid or a pharmaceutically acceptable salt thereof, and wherein the disorder is OCD, PTSD, or a phobia.

Another more specific embodiment of this invention relates to the above method wherein the compound administered is (3S, 4S)-(1-Aminomethyl-3,4-dimethyl-cyclopentyl)-acetic acid or a pharmaceutically acceptable salt thereof, and wherein the disorder is a phobia selected from agoraphobia and specific phobias.

Another more specific embodiment of the invention relates to the above method wherein the disorder being treated is fibromyalgia.

Another more specific embodiment of the invention relates to the above method for treating fibromyalgia, wherein the compound of formula 1 or 1A is (3S, 4S)-(1-aminomethyl-3,4-dimethyl-cyclopentyl)-acetic acid or a pharmaceutically acceptable salt thereof.

The invention also relates to a method for treating fibromyalgia and a concomitant disorder in a mammal, including a human, comprising administering to said mammal a therapeutically effective amount of a compound of formula 1 or 1A or a pharmaceutically acceptable salt thereof wherein said concomitant disorder is independently selected from migraine headaches, temporomandibular joint dysfunction, dysautonomia, endocrine dysfunction, dizziness, cold intolerance, chemical sensitivity, sicca symptoms, cognitive dysfunction, generalized anxiety disorder, premenstrual dysphoric dysthemia, irritable bowel syndrome, functional abdominal pain, neuropathic pain, somatoform disorders, OCD, phobias, and PTSD.

A more specific embodiment of this invention relates to the above method for treating fibromyalgia and a concomitant disorder wherein the compound administered is (3S, 4S)-(1-Aminomethyl-3,4-dimethyl-cyclopentyl)-acetic acid or a pharmaceutically acceptable salt thereof.

A more specific embodiment of this invention relates to the above method for treating fibromyalgia and a concomitant disorder wherein said concomitant disorder is generalized anxiety disorder, dysphoric dysthemia, irritable bowel syndrome, functional abdominal pain, neuropathic pain, a somatoform disorder, or migraine headache.

This invention also relates to a method of treating a disorder or condition selected from acute pain, chronic pain, pain resulting from soft tissue and peripheral damage such as acute trauma; complex regional pain syndrome also referred to as reflex sympathetic dystrophy; postherpetic neuralgia, occipital neuralgia, trigeminal neuralgia, segmental or intercostal neuralgia and other neuralgias; pain associated with osteoarthritis and rheumatoid arthritis; musculoskeletal pain such as pain associated with strains, sprains and trauma such as broken bones; spinal pain, central nervous system pain such as pain due to spinal cord or brain stem damage; lower back pain, sciatica, dental pain, myofascial pain syndromes, episiotomy pain, gout pain, and pain resulting from burns; deep and visceral pain, such as heart pain; muscle pain, eye pain, inflammatory pain, orofacial pain, for example, odontalgia; abdominal pain, and gynecological pain, for example, dysmenorrhoea, labour pain and pain associated with endometriosis; somatogenic pain; pain associated with nerve and root damage, such as pain associated with peripheral nerve disorders, for example, nerve entrapment, brachial plexus avulsions, and peripheral neuropathies ; pain associated with limb amputation, tic douloureux, neuroma, or vasculitis; diabetic neurapathy, chemotherapy-induced-neuropathy, acute herpetic and postherpetic neuralgia; atypical facial pain, nerve root damage, neuropathic lower back pain, HIV related neuropathic pain, cancer related neuropathic pain, diabetes related neuropathic pain and arachnoiditis, trigeminal neuralgia, occipital neuralgia, segmental or intercostal neuralgia, HIV related neuralgias and AIDS related neuralgias and other neuralgias; allodynia, hyperalgesia, burn pain, idiopathic pain, pain caused by chemotherapy; occipital neuralgia, psychogenic pain, brachial plexus avulsion, pain associated with restless legs syndrome; pain associated with gallstones; pain caused by chronic alcoholism or hypothyroidism or uremia or vitamin deficiencies; neuropathic and non-neuropathic pain associated with carcinoma, often referred to as cancer pain, phantom limb pain, functional abdominal pain, headache, including migraine with aura, migraine without aura and other vascular headaches, acute or chronic tension headache, sinus headache and cluster headache; temperomandibular pain and maxillary sinus pain; pain resulting from ankylosing spondylitis and gout; pain caused by increased bladder contractions; pain associated with gastrointestinal (GI) disorders, disorders caused by helicobacter pylori and diseases of the GI tract such as gastritis, proctitis, gastroduodenal ulcers, peptic ulcers, dyspepsia, disorders associated with the neuronal control of viscera, ulcerative colitis, chronic panceatitis, Crohn's disease and emesis; post operative pain, scar pain, and chronic non-neuropathic pain such as pain associated with HIV, anthralgia and myalgia, vasculitis and fibromyalgia in a mammal, comprising administering to a mammal in need of such treatment a therapeutically effective amount of a compound of formula 1 or 1A, or a pharmaceutically acceptable salt thereof.

This invention also relates to a method of treating a disorder or condition selected from the group consisting of mood disorders, such as depression, or more particularly, depressive disorders, for example, single episodic or recurrent major depressive disorder, severe unipolar recurrent major depressive episodes, dysthymic disorder, depressive neurosis and neurotic depression, melancholic depression including anorexia, weight loss, insomnia, early morning waking or psychomotor retardation, atypical depression (or reactive depression) including increased appetite, hypersomnia, psychomotor agitation or irritability; treatment resistant depression; seasonal affective disorder and pediatric depression; premenstrual syndrome, premenstrual dysphoric disorder, hot flashes, bipolar disorders or manic depression, for example, bipolar I disorder, bipolar II disorder and cyclothymic disorder; seasonal affective disorder, conduct disorder and disruptive behavior disorder; stress related somatic disorders and anxiety disorders, such as childhood anxiety disorder, panic disorder with or without agoraphobia, phobia including agoraphobia without history of panic disorder and specific phobias (e.g., specific animal phobias), social anxiety disorder, social phobia, obsessive-compulsive disorder(OCD), autism and associated disorders including pervasive developmental delay, mood disorders associated with psychotic disorders such as acute mania and depression associated with bipolar disorder, mood disorders associated with schizophrenia, behavioral disturbances associated with mental retardation, autistic disorder, conduct disorder and disruptive behavior disorder, borderline personality disorder, psychotic episodes of anxiety, and anxiety associated with psychosis; stress disorders including post-traumatic stress disorder (PTSD) and acute stress disorder, and generalized anxiety disorder in a mammal, comprising administering to a mammal in need of such treatment a therapeutically effective amount of therapeutically effective amount of a compound of formula 1 or 1A, or a pharmaceutically acceptable salt thereof.

It will be appreciated that for the treatment of depression or anxiety, a compound employed in the methods of the present invention may be used in conjunction with other antidepressant or anti-anxiety agents. Suitable classes of antidepressant agents include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists and atypical antidepressants. Suitable norepinephrine reuptake inhibitors include tertiary amine tricyclics and secondary amine tricyclics. Suitable examples of tertiary amine tricyclics include amitriptyline, clomipramine, doxepin, imipramine and trimipramine, and pharmaceutically acceptable salts thereof. Suitable examples of secondary amine tricyclics include amoxapine, desipramine, maprotiline, nortriptyline and protriptyline, and pharmaceutically acceptable salts thereof. Suitable selective serotonin reuptake inhibitors include fluoxetine, fluvoxamine, paroxetine and sertraline, and pharmaceutically acceptable salts thereof. Suitable monoamine oxidase inhibitors include isocarboxazid, phenelzine, tranylcypromine and selegiline, and pharmaceutically acceptable salts thereof. Suitable reversible inhibitors of monoamine oxidase include moclobemide, and pharmaceutically acceptable salts thereof. Suitable serotonin and noradrenaline reuptake inhibitors of use in the present invention include venlafaxine, and pharmaceutically acceptable salts thereof. Suitable CRF antagonists include those compounds described in International Patent Application Nos. WO 94/13643, WO 94/13644, WO 94/13661, WO 94/13676 and WO 94/13677. Suitable atypical anti-depressants include bupropion, lithium, nefazodone, trazodone and viloxazine, and pharmaceutically acceptable salts thereof. Suitable classes of anti-anxiety agents include benzodiazepines and 5-HT_{IA} agonists or antagonists, especially 5-HTIA partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam, and pharmaceutically acceptable salts thereof. Suitable 5-HT_{IA} receptor agonists or antagonists include, in particular, the 5-HT_{IA} receptor partial agonists buspirone, flesinoxan, gepirone and ipsapirone, and pharmaceutically acceptable salts thereof.

This invention also relates to a method of treating a disorder or condition selected from the group consisting of sleep disorders such as insomnia (e.g., primary insomnia including psychophysiological and idiopathic insomnia, secondary insomnia including insomnia secondary to restless legs syndrome, insomnia related to peri- and/or postmenopause, Parkinson's disease or another chronic disorder, and transient insomnia), somnambulism, sleep deprivation, REM sleep disorders, sleep apnea, hypersomnia, parasomnias, sleep-wake cycle disorders, jet lag, narcolepsy, sleep disorders associated with shift work or irregular work schedules, deficient sleep quality due to a decrease in slow wave sleep caused by medications or other sources, and other sleep disorders in a mammal, in a mammal, comprising administering to a mammal in need of such treatment a therapeutically effective amount of therapeutically effective amount of a compound of formula 1 or 1A, or a pharmaceutically acceptable salt thereof.

This invention also relates to a method of increasing slow wave sleep and increasing growth hormone secretion in a human subject in a mammal, comprising administering to a human subject in need of such treatment a therapeutically effective amount of therapeutically effective amount of a compound of formula 1 or 1A, or a pharmaceutically acceptable salt thereof.

This invention also relates to a method of treating a disorder or condition selected from the group consisting of respiratory diseases, particularly those associated with excess mucus secretion, such as chronic obstructive airways disease, bronchopneumonia, chronic bronchitis, cystic fibrosis, adult respiratory distress syndrome, and bronchospasm; cough, whooping cough, angiotensin converting enzyme (ACE) induced cough, pulmonary tuberculosis, allergies such as eczema and rhinitis; contact dermatitis, atopic dermatitis, urticaria, and other eczematoid dermatitis; itching, hemodialysis associated itching; inflammatory diseases such as inflammatory bowel disease, psoriasis, osteoarthritis, cartilage damage (e.g., cartilage damage resulting from physical activity or osteoarthritis), rheumatoid arthritis, psoriatic arthritis, asthma, pruritis and sunburn; and hypersensitivity disorders such as poison ivy in a mammal, including a human, comprising administering to a mammal in need of such treatment a therapeutically effective amount of therapeutically effective amount of a compound of formula 1 or 1A, or a pharmaceutically acceptable salt thereof.

Other more specific methods of this invention include any of the above methods wherein the compound of formula 1 or 1A is administered to a human for the treatment of any two or more comorbid disorders or conditions selected from those disorders and conditions referred to in any of the above methods.

Another more specific embodiment of this invention relates to any of the above methods for treating fibromyalgia wherein the compound of formula 1 or 1A is administered to a human for the treatment of fibromyalgia and concomitant generalized anxiety disorder.

Another more specific embodiment of this invention relates to any of the above methods wherein the compound of formula 1 or 1A is administered to a human for the treatment of major depressive disorder and concomitant irritable bowel syndrome.

Another more specific embodiment of this invention relates to any of the above methods wherein the compound of formula 1 or 1A is administered to a human for the treatment of major depressive disorder and concomitant functional abdominal pain.

Another more specific embodiment of this invention relates to any of the above methods wherein the compound of formula 1 or 1A is administered to a human for the treatment of major depressive disorder and concomitant neuropathic pain.

Another more specific embodiment of this invention relates to any of the above methods wherein the compound of formula 1 or 1A is administered to a human for the treatment of fibromyalgia and concomitant premenstrual dysphoric disorder.

Another more specific embodiment of this invention relates to any of the above methods wherein the compound of formula 1 or 1A is administered to a human for the treatment of major depressive disorder and concomitant dysthymia.

Another more specific embodiment of this invention relates to any of the above methods wherein the compound of formula 1 or 1A is administered to a human for the treatment of major depressive disorder and concomitant fibromyalgia.

Another more specific embodiment of this invention relates to any of the above methods wherein the compound of formula 1 or 1A is administered to a human for the treatment of dysthymia and concomitant fibromyalgia.

Another more specific embodiment of this invention relates to any of the above methods wherein the compound of formula 1 or 1A is administered to a human for the treatment of major depressive disorder and a concomitant somatoform disorder selected from somatization disorder, conversion disorder, body dysmorphic disorder, hypochondriasis, somatoform pain disorder, undifferentiated somatoform disorder and somatoform disorder not otherwise specified. See Diagnostic and Statistical manual of Mental Disorders, Fourth Edition (DSM-IV), American Psychiatric Association, Washington, D.C., May 1194, pp. 435-436.

Another more specific embodiment of this invention relates to any of the above methods wherein the compound of formula 1 or 1A is administered to a human for the treatment of fibromyalgia and concomitant irritable bowel syndrome.

Another more specific embodiment of this invention relates to any of the above methods wherein the compound of formula 1 or 1A is administered to a human for the treatment of fibromyalgia and concomitant functional abdominal pain.

Another more specific embodiment of this invention relates to any of the above methods wherein the compound of formula 1 or 1A is administered to a human for the treatment of fibromyalgia and concomitant neuropathic pain.

Another more specific embodiment of this invention relates to any of the above methods wherein the compound of formula 1 or 1A is administered to a human for the treatment of generalized anxiety disorder and concomitant premenstrual dysphoric disorder.

Another more specific embodiment of this invention relates to any of the above methods wherein the compound of formula 1 or 1A is administered to a human for the treatment of generalized anxiety disorder and concomitant dysthymia.

Another more specific embodiment of this invention relates to any of the above methods wherein the compound of formula 1 or 1A is administered to a human for the treatment of generalized anxiety disorder and concomitant fibromyalgia.

Another more specific embodiment of this invention relates to any of the above methods wherein the compound of formula 1 or 1A is administered to a human for the treatment of generalized anxiety disorder and a concomitant somatoform disorder selected from somatization disorder, conversion disorder, hypochondriasis, somatoform pain disorder (or simply “pain disorder”), body dysmorphic disorder, undifferentiated somatoform disorder, and somatoform disorder not otherwise specified. See Diagnostic and Statistical manual of Mental Disorders, Fourth Edition (DSM-IV), American Psychiatric Association, Washington, D.C., May 1194, pp. 435-436.

Another more specific embodiment of this invention relates to any of the above methods wherein the compound of formula 1 or 1A is administered to a human for the treatment of fibromyalgia and a concomitant somatoform disorder selected from somitization disorder, conversion disorder, hypochondriasis, somatoform pain disorder (or simply “pain disorder”), body dysmorphic disorder, undifferentiated somatoform disorder, and somatoform disorder not otherwise specified. See Diagnostic and Statistical manual of Mental Disorders, Fourth Edition (DSM-IV), American Psychiatric Association, Washington, D.C., May 1194, pp. 435-436.

Another more specific embodiment of this invention relates to any of the above methods wherein the compound of formula 1 or 1A is administered to a human for the treatment of major depressive disorder accompanied by one or more somatic symptoms selected from loss of appetite, sleep disturbances (e.g., insomnia, interrupted sleep, early morning awakening, tired awakening), loss of libido, restlessness, fatigue, constipation, dyspepsia, heart palpitations, aches and pains (e.g., headache, neck pain, back pain, limb pain, joint pain, abdominal pain), dizziness, nausea, heartburn, nervousness, tremors, burning and tingling sensations, morning stiffness, abdominal symptoms (e.g., abdominal pain, abdominal distention, gurgling, diarrhea), and the symptoms associated with generalized anxiety disorder (e.g., excessive anxiety and worry (apprehensive expectation), occurring more days than not for at least six months, about a number of events and activities, difficulty controlling the worry, etc.) See Diagnostic and Statistical manual of Mental Disorders, Fourth Edition (DSM-IV), American Psychiatric Association, Washington, D.C., May 1194, pp. 435-436 and 445-469. This document is incorporated herein by reference in its entirety.

Another more specific embodiment of this invention relates to any of the above methods wherein the formula 1 or 1A is administered to a human for the treatment of major depressive disorder accompanied by one or more somatic symptoms selected from fatigue, headache, neck pain, back pain, limb pain, joint pain, abdominal pain, abdominal distention, gurgling, diarrhea nervousness, and the symptoms associated with generalized anxiety disorder (e.g., excessive anxiety and worry (apprehensive expectation), occurring more days than not for at least six months, about a number of events and activities, difficulty controlling the worry, etc. See Diagnostic and Statistical manual of Mental Disorders, Fourth Edition (DSM-IV), American Psychiatric Association, Washington, D.C., May 1194, pp. 435-436 and 445-469.

Another more specific embodiment of this invention relates to any of the above methods wherein the compound of formula 1 or 1A is administered to a human for the treatment of generalized anxiety disorder accompanied by one or more somatic symptoms selected from loss of appetite, sleep disturbances (e.g., insomnia, interrupted sleep, early morning awakening, tired awakening), loss of libido, restlessness, fatigue, constipation, dyspepsia, heart palpitations, aches and pains (e.g., headache, neck pain, back pain, limb pain, joint pain, abdominal pain), dizziness, nausea, heartburn, nervousness, tremors, burning and tingling sensations, morning stiffness, abdominal symptoms (e.g., abdominal pain, abdominal distention, gurgling, diarrhea), and the symptoms associated with major depressive disorder (e.g., sadness, tearfulness, loss of interest, fearfulness, helplessness, hopelessness, fatigue, low self esteem, obsessive ruminations, suicidal thoughts, impaired memory and concentration, loss of motivation, paralysis of will, reduced appetite, increased appetite).

Another more specific embodiment of this invention relates to any of the above methods wherein the compound of formula 1 or 1A is administered to a human for the treatment of generalized anxiety disorder accompanied by one or more somatic symptoms selected from fatigue, headache, neck pain, back pain, limb pain, joint pain, abdominal pain, abdominal distention, gurgling, diarrhea nervousness, and the symptoms associated with major depressive disorder (e.g., sadness, tearfulness, loss of interest, fearfulness, helplessness, hopelessness, low self esteem, obsessive ruminations, suicidal thoughts, fatigue, impaired memory and concentration, loss of motivation, paralysis of will, reduced appetite, increased appetite).

This invention also relates to a method of treating a disorder or condition selected from the group consisting of sleep disorders such as insomnia (e.g., primary insomnia including psychophysiological and idiopathic insomnia, secondary insomnia including insomnia secondary to restless legs syndrome, Parkinson's disease or another chronic disorder, and transient insomnia), somnambulism, sleep deprivation, REM sleep disorders, sleep apnea, hypersomnia, parasomnias, sleep-wake cycle disorders, jet lag, narcolepsy, sleep disorders associated with shift work or irregular work schedules, deficient sleep quality due to a decrease in slow wave sleep caused by medications or other sources, and other sleep disorders in a mammal, comprising administering to a mammal in need of such treatment a therapeutically effective amount of a compound of the formula 1 or 1A, or a pharmaceutically acceptable salt thereof.

This invention also relates to a method of increasing slow wave sleep in a human subject comprising administering to a human subject in need of such treatment a therapeutically effective amount of a compound of the formula 1 or 1A or a pharmaceutically acceptable salt thereof.

This invention also relates to a method of increasing growth hormone secretion in a human subject comprising administering to a human subject in need of such treatment a therapeutically effective amount of a compound of the formula 1 or 1A or a pharmaceutically acceptable salt thereof.

This invention also relates to a method of increasing slow wave sleep in a human subject comprising administering to a human subject in need of such treatment:

(a) a compound of the formula 1 or 1A or a pharmaceutically acceptable salt thereof; and

(b) a human growth hormone or a human growth hormone secretagogue or a pharmaceutically acceptable salt thereof;

wherein the amounts of the active agents “a” and “b” are chosen so as to render the combination effective in increasing slow wave sleep.

A more specific embodiment of this invention relates to the above method wherein the human growth hormone secretagogue that is employed is 2-amino-N-[2-(3a-benzyl-2-methyl-3-oxo-2,3,3a,4,6,7-hexahydro-pyrazole[4,3-c]pyridin-5-yl)-1-benzyloxymethyl-2-oxo-ethyl]-2-methyl-proprionamide.

This invention also relates to a method of increasing slow wave sleep in a human subject being treated with an active pharmaceutical agent that decreases slow wave sleep, such as morphine or another opioid analgesic agent or a benzodiazepine, comprising administering to a human subject in need of such treatment:

(a) a compound of the formula 1 or 1A or a pharmaceutically acceptable salt thereof; and

(b) a human growth hormone or a human growth hormone secretagogue or a pharmaceutically acceptable salt thereof;

wherein the amounts of the active agents “a” and “b” are chosen so as to render the combination effective in increasing slow wave sleep.

A more specific embodiment of this invention relates to the above method wherein the human growth hormone secretagogue that is employed is 2-amino-N-[2-(3a-benzyl-2-methyl-3-oxo-2,3,3a,4,6,7-hexahydro-pyrazole[4,3-c]pyridin-5-yl)-1-benzyloxymethyl-2-oxo-ethyl]-2-methyl-proprionamide.

This invention also relates to a method of increasing slow wave sleep in a human subject being treated with an active pharmaceutical agent that decreases slow wave sleep, such as morphine or another opioid analgesic agent, comprising administering to such human subject an amount of a compound of the formula 1 or 1A, as defined above, or a pharmaceutically acceptable salt thereof, that is effective in increasing slow wave sleep.

This invention also relates to a method of treating irritable bowel syndrome in a mammal, preferably a human, comprising administering to a human subject in need of such treatment a therapeutically effective amount of a compound of the formula 1 or 1A, or a pharmaceutically acceptable salt thereof.

Preferred embodiments of the invention are the above methods that employ compounds of formula 1 wherein R¹ to R¹⁴ are selected from hydrogen, methyl, ethyl, propyl, isopropyl, butyl straight or branched, phenyl, or benzyl.

More preferred embodiments of the invention are the above methods that employ compounds of formula 1 wherein R¹ to R¹⁴ are selected from hydrogen, methyl, ethyl, or benzyl.

More specifically preferred embodiments of this invention are the above methods that employ compounds selected from:

(±)-(trans)-(1-Aminomethyl-3,4-dimethyl-cyclopentyl)-acetic acid hydrochloride;

(1-Aminomethyl-cyclobutyl)-acetic acid hydrochloride;

(cis/trans)-(3R)-(1-Aminomethyl-3-methyl-cyclopentyl)-acetic acid hydrochloride;

(cis)-(3R)-(1-Aminomethyl-3-methyl-cyclopentyl)-acetic acid hydrochloride;

(1α,3α4α)-(1-Aminomethyl-3,4-dimethyl-cyclopentyl)-acetic acid;

(1α,3α4α)-(1-Aminomethyl-3,4-diethyl-cyclopentyl)-acetic acid;

(1α,3α,4α)-(1-Aminomethyl-3,4-diisopropyl-cyclopentyl)-acetic acid;

[1S-(1α,3α,4α)]-(1-Aminomethyl-3-ethyl-4-methyl-cyclopentyl)-acetic acid;

[1R-(1α,3α,4α)]-(1-Aminomethyl-3-ethyl-4-methyl-cyclopentyl)-acetic acid;

[1S-(1α,3α,4α)]-(1-Aminomethyl-3-isopropyl-4-methyl-cyclopentyl)-acetic acid;

[1R-(1α,3α,4α)]-(1-Aminomethyl-3-isopropyl-4-methyl-cyclopentyl)-acetic acid;

[1S-(1α,3α,4α)]-(1-Aminomethyl-3-ethyl-4-isopropyl-cyclopentyl)-acetic acid;

[1R-(1α,3α,4α)]-(1-Aminomethyl-3-ethyl-4-isopropyl-cyclopentyl)-acetic acid;

[1S-(1α,3α,4α)]-(1-Aminomethyl-3-tert-butyl-4-methyl-cyclopentyl)-acetic acid;

[1R-(1α,3α,4α)]-(1-Aminomethyl-3-tert-butyl-4-methyl-cyclopentyl)-acetic acid;

[1S-(1α,3α4α)]-(1-Aminomethyl-3-tert-butyl-4-ethyl-cyclopentyl)-acetic acid;

[1R-(1α,3α,4α)]-(1-Aminomethyl-3-tert-butyl-4-ethyl-cyclopentyl)-acetic acid;

[1S-(1α,3α4α)]-(1-Aminomethyl-3-tert-butyl-4-isopropyl-cyclopentyl)-acetic acid;

[1R-(1α,3α,4α)]-(1-Aminomethyl-3-tert-butyl-4-isopropyl-cyclopentyl)-acetic acid;

(1α,3α,4α)-(1-Aminomethyl-3,4-di-tert-butyl-cyclopentyl)-acetic acid;

[1S-(1α,3α4α)]-(1-Aminomethyl-3-methyl-4-phenyl-cyclopentyl)-acetic acid;

[1R-(1α,3α4α)]-(1-Aminomethyl-3-methyl-4-phenyl-cyclopentyl)-acetic acid;

[1S-(1α,3α4α)]-(1-Aminomethyl-3-benzyl-4-methyl-cyclopentyl)-acetic acid;

[1R-(1α,3α,4α)]-(1-Aminomethyl-3-benzyl-4-methyl-cyclopentyl)-acetic acid;

(1S-cis)-(1-Aminomethyl-3-methyl-cyclopentyl)-acetic acid;

(1S-cis)-(1-Aminomethyl-3-ethyl-cyclopentyl)-acetic acid;

(1S-cis)-(1-Aminomethyl-3-isopropyl-cyclopentyl)-acetic acid;

(1S-cis)-(1-Aminomethyl-3-tert-butyl-cyclopentyl)-acetic acid;

(1S-cis)-(1-Aminomethyl-3-phenyl-cyclopentyl)-acetic acid;

(1S-cis)-(1-Aminomethyl-3-benzyl-cyclopentyl)-acetic acid;

(1R-cis)-(1-Aminomethyl-3-methyl-cyclopentyl)-acetic acid;

(1R-cis)-(1-Aminomethyl-3-ethyl-cyclopentyl)-acetic acid;

(1R-cis)-(1-Aminomethyl-3-isopropyl-cyclopentyl)-acetic acid;

(1R-cis)-(1-Aminomethyl-3-tert-butyl-cyclopentyl)-acetic acid;

(1R-cis)-(1-Aminomethyl-3-phenyl-cyclopentyl)-acetic acid;

(1R-cis)-(1-Aminomethyl-3-benzyl-cyclopentyl)-acetic acid;

(S)-(1-Aminomethyl-3,3-diethyl-cyclopentyl)-acetic acid;

(1-Aminomethyl-3,3,4,4-tetramethyl-cyclopentyl)-acetic acid;

(1-Aminomethyl-3,3,4,4-tetraethyl-cyclopentyl)-acetic acid;

(1α,3β,4β)-(1-Aminomethyl-3,4-dimethyl-cyclopentyl)-acetic acid;

(1α,3β,4β)-(1-Aminomethyl-3,4-diethyl-cyclopentyl)-acetic acid;

(1α,3β,4β)-(1-Aminomethyl-3,4-diisopropyl-cyclopentyl)-acetic acid;

[1R-(1α,3β,4β)]-(1-Aminomethyl-3-ethyl-4-methyl-cyclopentyl)-acetic acid;

[1S-(1α,3β,4β)]-(1-Aminomethyl-3-ethyl-4-methyl-cyclopentyl)-acetic acid;

[1R-(1α,3β,4β)]-(1-Aminomethyl-3-isopropyl-4-methyl-cyclopentyl)-acetic acid;

[1S-(1α,3β,4β)]-(1-Aminomethyl-3-isopropyl-4-methyl-cyclopentyl)-acetic acid;

[1R-(1α,3β,4β)]-(1-Aminomethyl-3-ethyl-4-isopropyl-cyclopentyl)-acetic acid;

[1S-(1α,3β,4β)]-(1-Aminomethyl-3-ethyl-4-isopropyl-cyclopentyl)-acetic acid;

[1R-(1α,3β,4β)]-(1-Aminomethyl-3-tert-butyl-4-methyl-cyclopentyl)-acetic acid;

[1S-(1α,3β,4β)]-(1-Aminomethyl-3-tert-butyl-4-methyl-cyclopentyl)-acetic acid;

[1R-(1α,3β,4β)]-(1-Aminomethyl-3-tert-butyl-4-ethyl-cyclopentyl)-acetic acid;

[1S-(1α,3β,4β)]-(1-Aminomethyl-3-tert-butyl-4-ethyl-cyclopentyl)-acetic acid;

[1R-(1α,3β,4β)]-(1-Aminomethyl-3-tert-butyl-4-isopropyl-cyclopentyl)-acetic acid;

[1S-(1α,3β,4β)]-(1-Aminomethyl-3-tert-butyl-4-isopropyl-cyclopentyl)-acetic acid;

(1α,3β,4β)-(1-Aminomethyl-3,4-di-tert-butyl-cyclopentyl)-acetic acid;

[1R-(1α,3β,4β)]-(1-Aminomethyl-3-methyl-4-phenyl-cyclopentyl)-acetic acid;

[1S-(1α,3β,4β)]-(1-Aminomethyl-3-methyl-4-phenyl-cyclopentyl)-acetic acid;

[1R-(1α,3β,4β)]-(1-Aminomethyl-3-benzyl-4-methyl-cyclopentyl)-acetic acid;

[1S-(1α,3β,4β)]-(1-Aminomethyl-3-benzyl-4-methyl-cyclopentyl)-acetic acid;

(1R-trans)-(1-Aminomethyl-3-methyl-cyclopentyl)-acetic acid;

(1R-trans)-(1-Aminomethyl-3-ethyl-cyclopentyl)-acetic acid;

(1R-trans)-(1-Aminomethyl-3-isopropyl-cyclopentyl)-acetic acid;

(1R-trans)-(1-Aminomethyl-3-tert-butyl-cyclopentyl)-acetic acid;

(1R-trans)-(1-Aminomethyl-3-phenyl-cyclopentyl)-acetic acid;

(1R-trans)-(1-Aminomethyl-3-benzyl-cyclopentyl)-acetic acid;

(1S-trans)-(1-Aminomethyl-3-methyl-cyclopentyl)-acetic acid;

(1S-trans)-(1-Aminomethyl-3-ethyl-cyclopentyl)-acetic acid;

(1S-trans)-(1-Aminomethyl-3-isopropyl-cyclopentyl)-acetic acid;

(1S-trans)-(1-Aminomethyl-3-tert-butyl-cyclopentyl)-acetic acid;

(1S-trans)-(1-Aminomethyl-3-phenyl-cyclopentyl)-acetic acid;

(1S-trans)-(1-Aminomethyl-3-benzyl-cyclopentyl)-acetic acid;

(R)-(1-Aminomethyl-3,3-diethyl-cyclopentyl)-acetic acid;

cis-(1-Aminomethyl-3-methyl-cyclobutyl)-acetic acid;

cis-(1-Aminomethyl-3-ethyl-cyclobutyl)-acetic acid;

cis-(1-Aminomethyl-3-isopropyl-cyclobutyl)-acetic acid;

cis-(1-Aminomethyl-3-tert-butyl-cyclobutyl)-acetic acid;

cis-(1-Aminomethyl-3-phenyl-cyclobutyl)-acetic acid;

trans-(1-Aminomethyl-3-methyl-cyclobutyl)-acetic acid;

trans-(1-Aminomethyl-3-ethyl-cyclobutyl)-acetic acid;

trans-(1-Aminomethyl-3-isopropyl-cyclobutyl)-acetic acid;

trans-(1-Aminomethyl-3-tert-butyl-cyclobutyl)-acetic acid;

trans-(1-Aminomethyl-3-phenyl-cyclobutyl)-acetic acid;

trans-(1-Aminomethyl-3-benzyl-cyclobutyl)-acetic acid;

cis-(1-Aminomethyl-3-ethyl-3-methyl-cyclobutyl)-acetic acid;

cis-(1-Aminomethyl-3-isopropyl-3-methyl-cyclobutyl)-acetic acid;

cis-(1-Aminomethyl-3-tert-butyl-3-methyl-cyclobutyl)-acetic acid;

cis-(1-Aminomethyl-3-methyl-3-phenyl-cyclobutyl)-acetic acid;

trans-(1-Aminomethyl-3-ethyl-3-methyl-cyclobutyl)-acetic acid;

trans-(1-Aminomethyl-3-isopropyl-3-methyl-cyclobutyl)-acetic acid;

trans-(1-Aminomethyl-3-tert-butyl-3-methyl-cyclobutyl)-acetic acid;

trans-(1-Aminomethyl-3-methyl-3-phenyl-cyclobutyl)-acetic acid;

cis-(1-Aminomethyl-3-ethyl-3-isopropyl-cyclobutyl)-acetic acid;

cis-(1-Aminomethyl-3-tert-butyl-3-ethyl-cyclobutyl)-acetic acid;

cis-(1-Aminomethyl-3-ethyl-3-phenyl-cyclobutyl)-acetic acid;

cis-(1-Aminomethyl-3-benzyl-3-ethyl-cyclobutyl)-acetic acid;

trans-(1-Aminomethyl-3-ethyl-3-isopropyl-cyclobutyl)-acetic acid;

trans-(1-Aminomethyl-3-tert-butyl-3-ethyl-cyclobutyl)-acetic acid;

trans-(1-Aminomethyl-3-ethyl-3-phenyl-cyclobutyl)-acetic acid;

trans-(1-Aminomethyl-3-benzyl-3-ethyl-cyclobutyl)-acetic acid;

cis-(1-Aminomethyl-3-tert-butyl-3-isopropyl-cyclobutyl)-acetic acid;

cis-(1-Aminomethyl-3-isopropyl-3-phenyl-cyclobutyl)-acetic acid;

trans-(1-Aminomethyl-3-benzyl-3-isopropyl-cyclobutyl)-acetic acid;

cis-(1-Aminomethyl-3-tert-butyl-3-phenyl-cyclobutyl)-acetic acid;

trans-(1-Aminomethyl-3-benzyl-3-tert-butyl-cyclobutyl)-acetic acid;

trans-(1-Aminomethyl-3-tert-butyl-3-isopropyl-cyclobutyl)-acetic acid;

trans-(1-Aminomethyl-3-isopropyl-3-phenyl-cyclobutyl)-acetic acid;

cis-(1-Aminomethyl-3-benzyl-3-isopropyl-cyclobutyl)-acetic acid;

trans-(1-Aminomethyl-3-tert-butyl-3-phenyl-cyclobutyl)-acetic acid;

cis-(1-Aminomethyl-3-benzyl-3-tert-butyl-cyclobutyl)-acetic acid;

(1-Aminomethyl-3,3-dimethyl-cyclobutyl)-acetic acid;

(1-Aminomethyl-3,3-diethyl-cyclobutyl)-acetic acid;

(1-Aminomethyl-3,3-diisopropyl-cyclobutyl)-acetic acid;

(1-Aminomethyl-3,3-di-tert-butyl-cyclobutyl)-acetic acid;

(1-Aminomethyl-3,3-diphenyl-cyclobutyl)-acetic acid;

(1-Aminomethyl-3,3-dibenzyl-cyclobutyl)-acetic acid;

(1-Aminomethyl-2,2,4,4-tetramethyl-cyclobutyl)-acetic acid;

(1-Aminomethyl-2,2,3,3,4,4-hexamethyl-cyclobutyl)-acetic acid;

(R)-(1-Aminomethyl-2,2-dimethyl-cyclobutyl)-acetic acid;

(S)-(1-Aminomethyl-2,2-dimethyl-cyclobutyl)-acetic acid;

(1R-cis)-(1-Aminomethyl-2-methyl-cyclobutyl)-acetic acid;

[1R-(1α,2α,3α)]-(1-Aminomethyl-2,3-dimethyl-cyclobutyl)-acetic acid;

(1α,2α,4α)-(1-Aminomethyl-2,4-dimethyl-cyclobutyl)-acetic acid;

[1R-(1α,2α,3β)]-(1-Aminomethyl-2,3-dimethyl-cyclobutyl)-acetic acid;

(1α,2α,4β)-(1-Aminomethyl-2,4-dimethyl-cyclobutyl)-acetic acid;

(1S-trans)-(1-Aminomethyl-2-methyl-cyclobutyl)-acetic acid;

[1S-(1α,2β,3β)]-(1-Aminomethyl-2,3-dimethyl-cyclobutyl)-acetic acid;

(1α,2β,4β)-(1-Aminomethyl-2,4-dimethyl-cyclobutyl)-acetic acid;

[1S-(1α,2β,3α)]-(1-Aminomethyl-2,3-dimethyl-cyclobutyl)-acetic acid;

(1α,2β,4α)-(1-Aminomethyl-2,4-dimethyl-cyclobutyl)-acetic acid;

(1R-trans)-(1-Aminomethyl-2-methyl-cyclobutyl)-acetic acid;

[1R-(1α,2β,3β)]-(1-Aminomethyl-2,3-dimethyl-cyclobutyl)-acetic acid;

[1R-(1α,2β,4β)]-(1-Aminomethyl-2-ethyl-4-methyl-cyclobutyl)-acetic acid;

[1R-(1α,2β,3α)]-(1-Aminomethyl-2,3-dimethyl-cyclobutyl)-acetic acid;

(1α,2β,4α)-(1-Aminomethyl-2,4-dimethyl-cyclobutyl)-acetic acid;

(1S-cis)-(1-Aminomethyl-2-methyl-cyclobutyl)-acetic acid;

[1S-(1α,2α,3α)]-(1-Aminomethyl-2,3-dimethyl-cyclobutyl)-acetic acid;

[1S-(1α,2α,3α)]-(1-Aminomethyl-2,4-dimethyl-cyclobutyl)-acetic acid;

[1S-(1α,2α,3α)]-(1-Aminomethyl-2,3-dimethyl-cyclobutyl)-acetic acid;

(1α,2α,4β)-(1-Aminomethyl-2,4-dimethyl-cyclobutyl)-acetic acid;

(3S, 4S)-(1-Aminomethyl-3,4-dimethyl-cyclopentyl)-acetic acid;

(3R, 4R)-(1-Aminomethyl-3,4-dimethyl-cyclopentyl)-acetic acid;

(3S, 4S))-(1-Aminomethyl-3,4-diethyl-cyclopentyl)-acetic acid;

(3R, 4R)-(1-Aminomethyl-3,4-diethyl-cyclopentyl)-acetic acid;

(3S, 4S)-(1-Aminomethyl-3,4-diisopropyl-cyclopentyl)-acetic acid;

(3R, 4R)-(1-Aminomethyl-3,4-diisopropyl-cyclopentyl)-acetic acid;

(3S, 4S)-(1-Aminomethyl-3,4-di-tert-butyl-cyclopentyl)-acetic acid;

(3R, 4R)-(1-Aminomethyl-3,4-di-tert-butyl-cyclopentyl)-acetic acid;

(3S, 4S)-(1-Aminomethyl-3,4-diphenyl-cyclopentyl)-acetic acid;

(3R, 4R)-(1-Aminomethyl-3,4-diphenyl-cyclopentyl)-acetic acid;

(3S, 4S)-(1-Aminomethyl-3,4-dibenzyl-cyclopentyl)-acetic acid;

(3R, 4R)-(1-Aminomethyl-3,4-dibenzyl-cyclopentyl)-acetic acid;

[1S-(1α,3α,4β)]-(1-Aminomethyl-3-methyl-4-ethyl-cyclopentyl)-acetic acid;

[1R-(1α,3β,4α)]-(1-Aminomethyl-3-methyl-4-ethyl-cyclopentyl)-acetic acid;

[1R-(1α,3α,4β)]-(1-Aminomethyl-3-methyl-4-ethyl-cyclopentyl)-acetic acid;

[1S-(1α,3β,4α)]-(1-Aminomethyl-3-methyl-4-ethyl-cyclopentyl)-acetic acid;

[1S-(1α,3α,4β)]-(1-Aminomethyl-3-methyl-4-isopropyl-cyclopentyl)-acetic acid;

[1R-(1α,3β,4α)]-(1-Aminomethyl-3-methyl-4-isopropyl-cyclopentyl)-acetic acid;

[1R-(1α,3α,4β)]-(1-Aminomethyl-3-methyl-4-isopropyl-cyclopentyl)-acetic acid;

[1S-(1α,3β,4α)]-(1-Aminomethyl-3-methyl-4-isopropyl-cyclopentyl)-acetic acid;

[1S-(1α,3α,4β)]-(1-Aminomethyl-3-methyl-4-tert-butyl-cyclopentyl)-acetic acid;

[1R-(1α,3β,4α)]-(1-Aminomethyl-3-methyl-4-tert-butyl-cyclopentyl)-acetic acid;

[1R-(1α,3α,4β)]-(1-Aminomethyl-3-methyl-4-tert-butyl-cyclopentyl)-acetic acid;

[1S-(1α,3β,4β)]-(1-Aminomethyl-3-methyl-4-tert-butyl-cyclopentyl)-acetic acid;

[1S-(1α,3α,4β)]-(1-Aminomethyl-3-methyl-4-phenyl-cyclopentyl)-acetic acid;

[1R-(1α,3β,4α)]-(1-Aminomethyl-3-methyl-4-phenyl-cyclopentyl)-acetic acid;

[1R-(1α,3α,4β)]-(1-Aminomethyl-3-methyl-4-phenyl-cyclopentyl)-acetic acid;

[1S-(1α,3β,4α)]-(1-Aminomethyl-3-methyl-4-phenyl-cyclopentyl)-acetic acid;

[1S-(1α,3α,4β)]-(1-Aminomethyl-3-benzyl-4-methyl-cyclopentyl)-acetic acid;

[1R-(1α,3β,4α)]-(1-Aminomethyl-3-benzyl-4-methyl-cyclopentyl)-acetic acid;

[1R-(1α,3α,4β)]-(1-Aminomethyl-3-benzyl-4-methyl-cyclopentyl)-acetic acid;

[1S-(1α,3β,4α)]-(1-Aminomethyl-3-benzyl-4-methyl-cyclopentyl)-acetic acid;

[1S-(1α,3α,4β)]-(1-Aminomethyl-3-ethyl-4-isopropyl-cyclopentyl)-acetic acid;

[1R-(1α,3β,4α)]-(1-Aminomethyl-3-ethyl-4-isopropyl-cyclopentyl)-acetic acid;

[1R-(1α,3α,4β)]-(1-Aminomethyl-3-ethyl-4-isopropyl-cyclopentyl)-acetic acid;

[1S-(1α,3β,4α)]-(1-Aminomethyl-3-ethyl-4-isopropyl-cyclopentyl)-acetic acid;

[1S-(1α,3α,4β)]-(1-Aminomethyl-3-tert-butyl-4-ethyl-cyclopentyl)-acetic acid;

[1R-(1α,3β,4α)]-(1-Aminomethyl-3-tert-butyl-4-ethyl-cyclopentyl)-acetic acid;

[1R-(1α,3α,4β)]-(1-Aminomethyl-3-tert-butyl-4-ethyl-cyclopentyl)-acetic acid;

[1S-(1α,3β,4α)]-(1-Aminomethyl-3-tert-butyl-4-ethyl-cyclopentyl)-acetic acid;

[1S-(1α,3α,4β)]-(1-Aminomethyl-3-ethyl-4-phenyl-cyclopentyl)-acetic acid;

[1R-(1α,3β,4α)]-(1-Aminomethyl-3-ethyl-4-phenyl-cyclopentyl)-acetic acid;

[1R-(1α,3α,4β)]-(1-Aminomethyl-3-ethyl-4-phenyl-cyclopentyl)-acetic acid;

[1S-(1α,3β,4α)]-(1-Aminomethyl-3-ethyl-4-phenyl-cyclopentyl)-acetic acid;

[1S-(1α,3α,4β)]-(1-Aminomethyl-3-benzyl-4-ethyl-cyclopentyl)-acetic acid;

[1R-(1α,3β,4α)]-(1-Aminomethyl-3-benzyl-4-ethyl-cyclopentyl)-acetic acid;

[1R-(1α,3α,4β)]-(1-Aminomethyl-3-benzyl-4-ethyl-cyclopentyl)-acetic acid;

[1S-(1α,3β,4α)]-(1-Aminomethyl-3-benzyl-4-ethyl-cyclopentyl)-acetic acid;

[1S-(1α,3α,4β)]-(1-Aminomethyl-3-tert-butyl-4-isopropyl-cyclopentyl)-acetic acid;

[1R-(1α,3β,4α)]-(1-Aminomethyl-3-tert-butyl-4-isopropyl-cyclopentyl)-acetic acid;

[1R-(1α,3α,4β)]-(1-Aminomethyl-3-tert-butyl-4-isopropyl-cyclopentyl)-acetic acid;

[1S-(1α,3β,4α)]-(1-Aminomethyl-3-tert-butyl-4-isopropyl-cyclopentyl)-acetic acid;

[1S-(1α,3α,4β)]-(1-Aminomethyl-3-isopropyl-4-phenyl-cyclopentyl)-acetic acid;

[1R-(1α,3β,4α)]-(1-Aminomethyl-3-isopropyl-4-phenyl-cyclopentyl)-acetic acid;

[1R-(1α,3α,4β)]-(1-Aminomethyl-3-isopropyl-4-phenyl-cyclopentyl)-acetic acid;

[1S-(1α,3β,4α)]-(1-Aminomethyl-3-isopropyl-4-phenyl-cyclopentyl)-acetic acid;

[1S-(1α,3α,4β)]-(1-Aminomethyl-3-benzyl-4-isopropyl-cyclopentyl)-acetic acid;

[1R-(1α,3β,4α)]-(1-Aminomethyl-3-benzyl-4-isopropyl-cyclopentyl)-acetic acid;

[1R-(1α,3α,4β)]-(1-Aminomethyl-3-benzyl-4-isopropyl-cyclopentyl)-acetic acid;

[1S-(1α,3β,4α)]-(1-Aminomethyl-3-benzyl-4-isopropyl-cyclopentyl)-acetic acid;

[1S-(1α,3α,4β)]-(1-Aminomethyl-3-tert-butyl-4-phenyl-cyclopentyl)-acetic acid;

[1R-(1α,3β,4α)]-(1-Aminomethyl-3-tert-butyl-4-phenyl-cyclopentyl)-acetic acid;

[1R-(1α,3α,4β)]-(1-Aminomethyl-3-tert-butyl-4-phenyl-cyclopentyl)-acetic acid;

[1S-(1α,3β,4α)]-(1-Aminomethyl-3-tert-butyl-4-phenyl-cyclopentyl)-acetic acid;

[1R-(1α,3α,4β)]-(1-Aminomethyl-3-benzyl-4-tert-butyl-cyclopentyl)-acetic acid;

[1S-(1α,3β,4α)]-(1-Aminomethyl-3-benzyl-4-tert-butyl-cyclopentyl)-acetic acid;

[1S-(1α,3α,4β)]-(1-Aminomethyl-3-benzyl-4-tert-butyl-cyclopentyl)-acetic acid;

[1R-(1α,3β,4α)]-(1-Aminomethyl-3-benzyl-4-tert-butyl-cyclopentyl)-acetic acid;

[1S-(1α,3α,4β)]-(1-Aminomethyl-3-benzyl-4-phenyl-cyclopentyl)-acetic acid;

[1R-(1α,3β,4α)]-(1-Aminomethyl-3-benzyl-4-phenyl-cyclopentyl)-acetic acid;

[1R-(1α,3α,4β)]-(1-Aminomethyl-3-benzyl-4-phenyl-cyclopentyl)-acetic acid;

[1S-(1α,3β,4α)]-(1-Aminomethyl-3-benzyl-4-phenyl-cyclopentyl)-acetic acid;

(1R-cis)-(1-Aminomethyl-2-methyl-cyclopentyl)-acetic acid;

(1S-cis)-(1-Aminomethyl-2-methyl-cyclopentyl)-acetic acid;

(1R-trans)-(1-Aminomethyl-2-methyl-cyclopentyl)-acetic acid;

(1S-trans)-(1-Aminomethyl-2-methyl-cyclopentyl)-acetic acid;

(R)-(1-Aminomethyl-2,2-dimethyl-cyclopentyl)-acetic acid;

(S)-(1-Aminomethyl-2,2-dimethyl-cyclopentyl)-acetic acid;

(1-Aminomethyl-2,2,5,5-tetramethyl-cyclopentyl)-acetic acid;

(1α,2β,5β)-(1-Aminomethyl-2,5-dimethyl-cyclopentyl)-acetic acid;

(2R, 5R)-(1-Aminomethyl-2,5-dimethyl-cyclopentyl)-acetic acid;

(2S. 5S)-(1-Aminomethyl-2,5-dimethyl-cyclopentyl)-acetic acid;

(1α,2α,5α)-(1-Aminomethyl-2,5-dimethyl-cyclopentyl)-acetic acid;

[1R-(1α,2α,3α)]-(1-Aminomethyl-2,3-dimethyl-cyclopentyl)-acetic acid;

[1R-(1α,2β,3α)]-(1-Aminomethyl-2,3-dimethyl-cyclopentyl)-acetic acid;

[1R-(1α,2α,3β)]-(1-Aminomethyl-2,3-dimethyl-cyclopentyl)-acetic acid;

[1R-(1α,2β,3β)]-(1-Aminomethyl-2,3-dimethyl-cyclopentyl)-acetic acid;

[1S-(1α,2α,3α)]-(1-Aminomethyl-2,3-dimethyl-cyclopentyl)-acetic acid;

[1S-(1α,2β,3α)]-(1-Aminomethyl-2,3-dimethyl-cyclopentyl)-acetic acid;

[1S-(1α,2α,3β)]-(1-Aminomethyl-2,3-dimethyl-cyclopentyl)-acetic acid;

[1S-(1α,2β,3β)]-(1-Aminomethyl-2,3-dimethyl-cyclopentyl)-acetic acid;

[1R-(1α,2α,4α)]-(1-Aminomethyl-2,4-dimethyl-cyclopentyl)-acetic acid;

[1S-(1α,2α,4α)]-(1-Aminomethyl-2,4-dimethyl-cyclopentyl)-acetic acid;

[1R-(1α,2α,4β)]-(1-Aminomethyl-2,4-dimethyl-cyclopentyl)-acetic acid;

[1S-(1α,2α,4β)]-(1-Aminomethyl-2,4-dimethyl-cyclopentyl)-acetic acid;

[1R-(1α,2β,4α)]-(1-Aminomethyl-2,4-dimethyl-cyclopentyl)-acetic acid;

[1S-(1α,2β,4α)]-(1-Aminomethyl-2,4-dimethyl-cyclopentyl)-acetic acid;

[1R-(1α,2β,4β)]-(1-Aminomethyl-2,4-dimethyl-cyclopentyl)-acetic acid; and

[1S-(1α,2β,4β)]-(1-Aminomethyl-2,4-dimethyl-cyclopentyl)-acetic acid;

(trans)-(3,4-Dimethyl-cyclopentylidene)-acetic acid ethyl ester;

(trans)-(3,4-Dimethyl-1-nitromethyl-cyclopentyl)-acetic acid;

(±)-(trans)-7,8-Dimethyl-2-aza-spiro[4.4]nonane-2-one;

(1-Nitromethyl-cyclobutyl)-acetic acid ethyl ester;

(cis/trans)-(3R)-(3-Methyl-1-nitromethyl-cyclopentyl)-acetic acid ethyl ester;

(cis/trans)-(7R)-7-Methyl-2-aza-spiro[4.4]nonane-2-one;

(cis)-(3,4-Dimethyl-cyclopentyldiene)-acetic acid ethyl ester;

(trans)-3,4-Dimethyl-1-nitromethyl-cyclopentyl)-acetic acid ethyl ester;

(trans)-7,8-Dimethyl-2-aza-spiro[4.4]nonane-2-one;

(3-Benzyl-cyclobutylidene)-acetic acid ethyl ester; and

(cis/trans)-(3-Benzyl-1-nitromethyl-cyclopentyl)-acetic acid ethyl ester.

Especially preferred embodiments of this invention relate to any of the above methods wherein the compound being administered is (3S, 4S)-(1-Aminomethyl-3,4-dimethyl-cyclopentyl)-acetic acid.

The term “lower alkyl” is a straight or branched group of from 1 to 4 carbons.

The term “alkyl” is a straight or branched group of from 1 to 6 carbon atoms including but not limited to methyl, ethyl, propyl, n-propyl, isopropyl, butyl, 2-butyl, tert-butyl, pentyl, except as where otherwise stated.

The benzyl and phenyl groups of compounds of the formulas 1 and 1A may be unsubstituted or substituted by from 1 to 3 substituents selected from hydroxy, carboxy, carboalkoxy, halogen, CF₃, nitro, alkyl, and alkoxy. Preferred are halogens.

Since amino acids are amphoteric, pharmacologically compatible salts when R is hydrogen can be salts of appropriate inorganic or organic acids, for example, hydrochloric, sulphuric, phosphoric, acetic, oxalic, lactic, citric, malic, salicylic, malonic, maleic, succinic, methanesulfonic acid, and ascorbic. Starting from corresponding hydroxides or carbonates, salts with alkali metals or alkaline earth metals, for example, sodium, potassium, magnesium, or calcium are formed. Salts with quaternary ammonium ions can also be prepared with, for example, the tetramethyl-ammonium ion. The carboxyl group of the amino acids can be esterified by known means.

Certain of the compounds employed in the methods of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds employed in the methods of the present invention possess one or more chiral centers and each center may exist in the R(D) or S(L) configuration. The present invention includes all enantiomeric and epimeric forms as well as the appropriate mixtures thereof.

One benefit of using the compounds of this invention to treat fibromyalgia is that they are not addictive. In these methods, the compounds can be combined with other agents including antidepressant and/or anti-anxiety agents.

### DETAILED DESCRIPTION OF THE INVENTION

Compounds of the formulas 1 and 1A can be prepared as described below and in U.S. patent application Ser. No. 09/485,382 filed Feb. 8, 2000.

Both the 4- and 5-membered ring compounds of formulas 1 and 1A may be synthesized by the routes outlined below for the 5-membered ring system. The compounds of formulas 1 and 1A may be synthesized, for example, by utilizing the general strategy (General Scheme 1) outlined by G. Griffiths et al., Helv. Chim. Acta, 1991;74:309. Alternatively, they may also be made as shown (General Scheme 2), analogously to the published procedure for the synthesis of 3-oxo-2,8-diazaspiro[4,5]decane-8-carboxylic acid tert-butyl ester (P. W. Smith et al., J. Med. Chem., 1995;38:3772). The compounds may also be synthesized by the methods outlined by G. Satzinger et al., (Ger Offen 2,460,891; U.S. Pat. No. 4,024,175, and Ger Offen 2,611,690; U.S. Pat. No. 4,152,326) (General Schemes 3 and 4). The compounds may also be synthesized by the route outlined by G. Griffiths et al., Helv. Chim. Acta, 1991;74:309 (General Scheme 5).

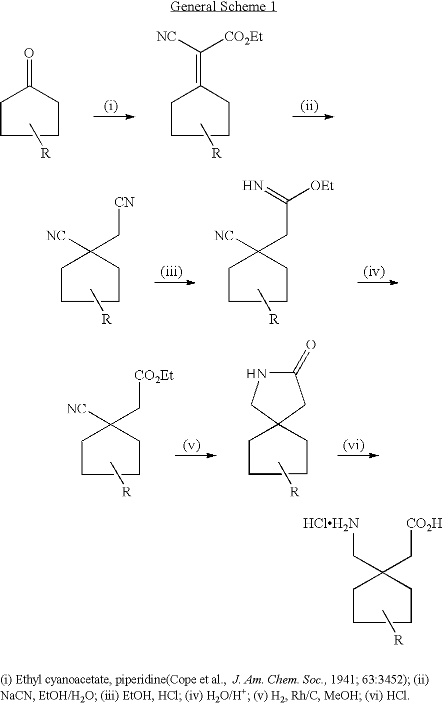

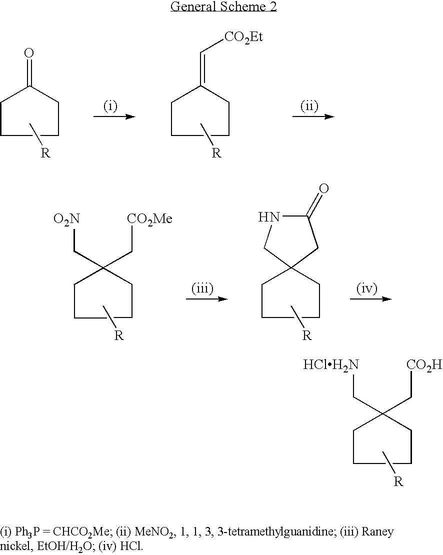

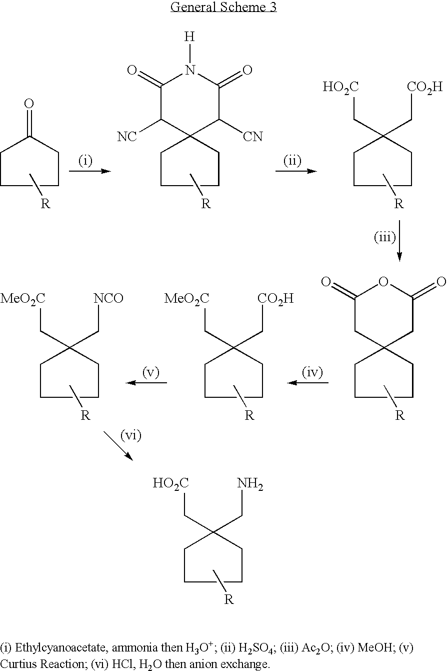

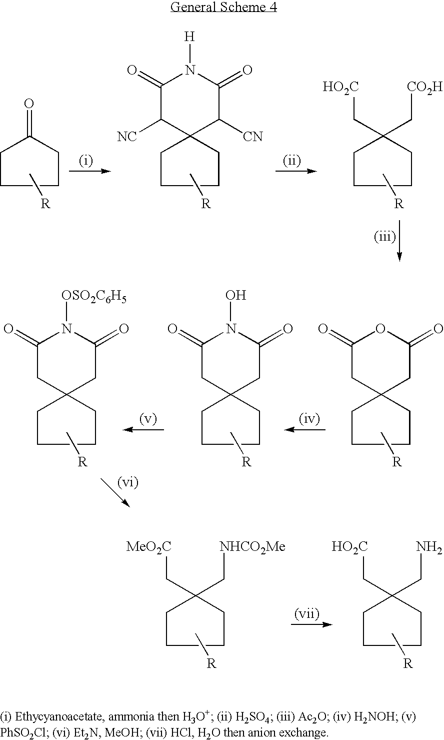

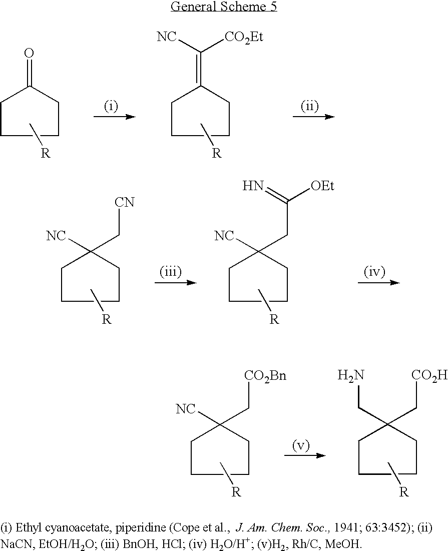

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally. It will be obvious to those skilled in the art that the following dosage forms may comprise as the active component, either a compound of formula 1 or 1A or a corresponding pharmaceutically acceptable salt of a compound of formula 1 or 1A.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted, and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection liquid preparations can be formulated in solution in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1 g according to the particular application and the potency of the active component. In medical use the drug may be administered three times daily as, for example, capsules of 100 or 300 mg. The composition can, if desired, also contain other compatible therapeutic agents.

In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

The anxiolytic and antidepressant activity of (3S, 4S)-(1-Aminomethyl-3,4-dimethyl-cyclopentyl)-acetic acid (“Compound A”) was assessed using the Tail Suspension Test (TST) in mice, and in the Water-lick (Vogel) Conflict Test (WLC) in rats. The Vogel test is a recognized test procedure for assessing the potential anxiolytic utility. The TST procedure is a behavior despair paradigm used to assess potential antidepressant activity.

Tail Suspension Test

The method consisted of suspending mice by a piece of cellophane tape attached to the distal end of the tail for 6 minutes. Animals (CD-1 mice, weighing 22 to 27 g, from Charles River Labs) were tested. The test apparatus was the TST-2™ (ITEM-Labo, Le Kremmlin-Bicetre Cedex, France). Data were analyzed with analysis of variance (ANOVA) and Tukey's Multiple Range Test or Student's t-test.

Immediately following the TST, mice were also tested in the Inverted Screen Test to assess coordination. Animals were given a 1-minute trial in which they had to climb to the top of the inverted screen or to simply hang on and not fall off.

Water-lick Conflict Test

In each experiment, naive adult male Wistar rats between 170 to 200 g were randomly divided into groups (N=10-30/group) and deprived of water for 48 hours prior to testing. Food was available on Day 1 and removed 24 hours prior to test Day 2.

Apparatus: The modular operant test cage (Coulbourn Instruments) measures 10.25×12×12 in. The test chambers feature 3 modular bays on each side of the cage for a total of 6 bays. A module optical lickometer is mounted on one side of the chamber 5 cm above the grid floor. The lickometer is used to measure licking-drinking from a water bottle mounted outside the test chamber. A photo beam is piped via glass rods to the tip of the drink tube across a gap at the end of the tube. The animals tongue breaks the beam on each lick. The front and back of the test chamber is made of clear Plexiglas. The front door is covered to reduce distractions from inside the test room. The back of the test chamber is facing a wall, away from the flow of traffic within the testing room and remains uncovered to provide the opportunity for observations during testing. Shocks are delivered using a (Coulbourn) programmable universal shocker calibrated to deliver a 1 mA shock for 1 second through the drink tube.

Procedure: On Day 1, after a 24-hour water deprivation, experimental subjects were placed into the test chambers and allowed to drink unpunished. Drinking was limited to 500 responses or approximately 5 mL of water during a 10 minute session. Immediately following the unpunished drinking session, rats were returned to their home cages, deprived of water for an additional 24 hours and was food deprived. On test Day 2, rats were dosed with vehicle or Compound A orally (PO) 120 minutes prior to testing in the Water-Lick (Vogel) Conflict test. After the pretreatment period rats were placed into a test chamber and allowed to drink for 10 minutes. After every 10 licks, rats received a 1-second shock (1 mA) through the drink tube. Thus, a conflict or anxiety-producing situation exists. Rats are motivated to drink; however, they are inhibited by the shock. Anxiety is reflected by the low amounts of drinking. Standard anxiolytic drugs produce effects that allow rats to overcome this behavioral inhibition and drink despite the shock. Compounds that significantly increase the number of shock episodes over concurrently run controls are presumed to possess anxiolytic-like properties.

All data were analyzed using a Kruskal-Wallis one Way Analysis of Variance on Ranks and Mann-Whitney Rank Sum Tests.

Quantitative Analysis: A quantitative analysis represents the percentage of subjects within a treatment group that receives >20 shock episodes during a test session. This number provides a quantitative comparison regarding the distribution of the responses.

Compound A was dissolved in water and tested orally as a solution from 0.3 to 100 mg/kg in rats and 3 to 300 mg/kg in mice. Dosages are expressed as the active moiety and were administered in a volume of 1 mL/kg for rats and 10 mL/kg for mice.

The profile of typical anxiolytic-like activity in the TST consists of increased immobility while the power of movement is diminished. Compound A and pregabalin were tested concurrently (PO) 2 hours after treatment. Compound A was administered at doses ranging from 3 to 100 mg/kg and pregabalin was tested from 3-300 mg/kg and served as positive control (Table 1). Compound A dose dependently increases immobility with the MED observed at the 3 mg/kg dosage and maximal effects were seen following the 30 mg/kg dose. The power of movement parameter was decreased at the 30 and 100 mg/kg dosages for Compound A which are doses 10 and 30× the MED for increasing immobility.

In the Inverted Screen Test, Compound A did not cause animals to fall off at doses up to 100 mg/kg which is 30× the TST MED. Pregabalin produced screen fall-off in 1 of 10 animals tested at the 100 and 300 mg/kg dosages.

In the Water-lick (Vogel) Conflict Test, Compound A produced significant anti-conflict activity across a wide range of oral doses following a 2 hour pretreatment similar to pregabalin (Table 2). The MED for Compound A was seen at the 3 mg/kg dosage and the maximal effects were observed following the 100 mg/kg dose. The magnitude of this response is similar to pregabalin 10 mg/kg (Table 2).

Time-course effects for Compound A demonstrated the onset of the anxiolytic-like activity as well as the duration of action of anti-conflict activity when compared to currently run controls. The onset of activity for Compound A was observed beginning 2 hours after treatment and was maintained through the 6 hours, peak activity was observed at the 2 hour time-point. The onset of activity for doses 3× and 10× the Vogel MED (10 and 30 mg/kg) activity was observed beginning 1 hour after treatment and was maintained through the 6 hour time-point. Peak activity was seen between 4-6 hours after treatment respectively (Table 3).

For comparison, pregabalin was tested under similar experimental conditions. The MED and onset of activity for pregabalin were shifted to the right on the dose response curve. The MED for pregabalin was 10 mg/kg and the maximal effects were observed 2-4 hours after treatment. The onset of activity for a dose 3× the Vogel MED (30 mg/kg) was observed 1 hour after treatment and activity was maintained through 8 hours. Peak activity was seen 6 hours post treatment (Table 4).

TABLE 1
Dose response effects of Compound A compared to
pregabalin in the Tail Suspension Test 2 hours
after treatment in mice.

| | | | Number | % Percent of control | |
|---|---|---|---|---|---|
| | Dose | | of mice/ | | Power of |
| Treatment | (mg/kg) | Route | group | Immobility | Movement |
| Compound A | 3 | PO | 10 | 126.8 ± 7.0* | 70.7 ± 10.3 |
| | 10 | PO | 10 | 143.7 ± 11.0* | 60.8 ± 11.4 |
| | 30 | PO | 10 | 185.9 ± 7.0* | 46.4 ± 12.3* |
| | 100 | PO | 10 | 182.4 ± 5.9* | 50.5 ± 18.3* |
| Pregabalin | 3 | PO | 10 | 102.9 ± 9.5 | 99.1 ± 22.9 |
| | 10 | PO | 10 | 134.5 ± 9.9* | 89.6 ± 18.7 |
| | 30 | PO | 10 | 136.9 ± 7.1* | 96.4 ± 22.7 |
| | 100 | PO | 10 | 152.7 ± 6.6* | 73.4 ± 12.5 |
| | 300 | PO | 10 | 160.5 ± 7.4* | 76.6 ± 12.2 |

Summary effects of Compound A compared to pregabalin in the Tail Suspension Test in Mice (Bold equals MED TST).
*p < 0.05 relative to vehicle-treated controls. T-Test

TABLE 2
Effect on Conflict Behavior: Dose response effects of Compound A
compared to pregabalin (PO) in the Water-lick (Vogel) Conflict Test in rats.

| | | | | | | Percent of |
|---|---|---|---|---|---|---|
| | | | | Pre- | Mean | Animals |
| | | | | treatment | Shock | Tested With |
| | Dose | | Number | time | Episodes | >20 Shock |
| Treatment | (mg/kg) | Route | of rats/group | minutes | (1 mA) | Episodes |
| Vehicle | 0 | PO | 30 | 120 | 6.8 ± 2.14 | 7% |
| Compound A | 0.3 | PO | 10 | 120 | 16.5 ± 8.7 | 20% |
| | 1 | PO | 30 | 120 | 11.7 ± 4.2 | 10% |
| | 3 | PO | 30 | 120 | 16.7 ± 4.3* | 17% |
| | 10 | PO | 30 | 120 | 33.8 ± 6.2* | 43% |
| | 30 | PO | 30 | 120 | 46.3 ± 7.6* | 67% |
| | 100 | PO | 10 | 120 | 52.8 ± 10.9* | 90% |
| Vehicle | 0 | PO | 20 | 120 | 3.9 ± 0.4 | 0% |
| Pregabalin | 0.3 | PO | 10 | 120 | 5.1 ± 1.0 | 0% |
| | 1 | PO | 10 | 120 | 15.0 ± 6.3 | 20% |
| | 3 | PO | 20 | 120 | 10.2 ± 1.6 | 20% |
| | 10 | PO | 10 | 120 | 14.7 ± 4.0* | 20% |
| | 30 | PO | 10 | 120 | 51.0 ± 19.1* | 50% |
| | 100 | PO | 10 | 120 | 40.7 ± 11.1* | 50% |

The effect of Compound A compared to pregabalin (PO) in the Water-lick Conflict test in rats (Bold equals MED in WLC).
Data are Mean ± SEM
*p < 0.05, vs vehicle, Kruskal-Wallis One Way Analysis of Variance on Ranks Mann-Whitney Rank Sum Test

TABLE 3
Time-course effects for Compound A, the MED (3 mg/kg) and doses 3X and
10X the Vogel MED (10 and 30 mg/kg) in the Water-Lick (Vogel) Conflict Test in rats

| | Time post treatment | | | | | |
|---|---|---|---|---|---|---|
| | 30 min. | 1 hr | 2 hr | 4 hr | 6 hr | 8 hr |
| TREATMENT | Shock episodes | | | | | |
| Vehicle | — | 28.1 ± 10.6 | 18.3 ± 7.5 | 9.2 ± 2.9 | 10.7 ± 3.6 | 20.9 ± 7.2 |
| Compound A | — | 38.8 ± 12.2 | 55.7 ± 12.7* | 38.4 ± 12.2* | 44.7 ± 13.3* | 7.7 ± 1.2 |
| 3 mg/kg | | | | | | |
| (MED) | | | | | | |
| Vehicle | 20.3 ± 10.8 | 13.0 ± 7.1 | 4.4 ± 1.3 | 4.8 ± 0.8 | 25.2 ± 10.6 | — |
| Compound A | 14.1 ± 3.1 | 28.2 ± 7.6* | 29.6 ± 8.0* | 47.7 ± 11.7* | 38.4 ± 13.2 | — |
| 10 mg/kg | | | | | | |
| (3X MED) | | | | | | |
| Vehicle | 20.4 ± 12.2 | 21.9 ± 8.3 | 11.6 ± 2.9 | 9.5 ± 4.1 | 7.0 ± 2.2 | — |
| Compound A | 35.8 ± 12.6 | 93.3 ± 12.1* | 88.4 ± 7.4* | 73.5 ± 12.2* | 97.4 ± 11.1* | — |
| 30 mg/kg | | | | | | |
| (10X MED) | | | | | | |

Time-course effects for Compound A in the Water-Lick Conflict Test in rats, N = 10/group (Bold equals MED in WLC).
*p < 0.05 vs currently run vehicle One Way ANOVA

TABLE 4
Time-course effects for Pregabalin, the MED (10 mg/kg) and 3X the MED
(30 mg/kg) in the Water-Lick (Vogel) Conflict Test in rats

| | Time post treatment | | | | | |
|---|---|---|---|---|---|---|
| | 30 min. | 1 hr | 2 hr | 4 hr | 6 hr | 8 hr |
| TREATMENT | Shock episodes | | | | | |
| Vehicle | — | 12.6 ± 2.9 | 23.9 ± 10.0 | 14.3 ± 7.6 | 23.1 ± 10.4 | 16.1 ± 6.5 |
| Pregabalin | — | 65.8 ± 10.8 | 73.3 ± 13.7* | 75.1 ± 13.7* | 54.9 ± 14.7* | 41.9 ± 13.5 |
| 10 mg/kg | | | | | | |
| Vehicle | 9.0 ± 1.8 | 14.9 ± 3.4 | 7.3 ± 1.1 | 9.8 ± 1.4 | 5.3 ± 0.9 | 5.8 ± 0.7 |
| Pregabalin | 27.7 ± 8.2 | 66.5 ± 10* | 59.8 ± 9.8* | 100.3 ± 12* | 39.5 ± 9* | 30.4 ± 9.1* |
| 30 mg/kg | | | | | | |

Pregabalin time-course effects in the Water-lick Conflict test (10 and 30 mg/kg) in rats. N = 10/group, (Bold equals MED in WLC)
*p < 0.05 vs currently run vehicle One Way ANOVA

(3S, 4S)-(1-Aminomethyl-3,4-dimethyl-cyclopentyl)-acetic acid (“Compound A”) was further evaluated using:

### A RAT MODEL OF FOOTPAD TACTILE ALLODYNIA FROM PRIOR INJECTION OF ACID INTO THE GASTROCNEMIUS MUSCLE

Patients with fibromyalgia syndrome (FMS) typically demonstrate widespread, chronic musculoskeletal pain, which is often accompanied by tactile allodynia (pain in response to a relatively light tactile stimulus that is normally not painful). A rat model of persistent mechanical allodynia has been developed that is consistent with the muscle tenderness found in these patients. Multiple injections of acidified saline into the gastrocnemius muscle in rats produce a long- lasting allodynia (conveniently measured at the footpad) that is thought to be centrally mediated (Sluka K, Kalra A, Moore S. Unilateral intramuscular injections of acidic saline produce a bilateral, long-lasting hyperalgesia. Muscle Nerve 2001;24:37-46; Sluka K, Rohlwing J, Bussey R, et al. Chronic muscle pain induced by repeated acid injection is reversed by spinally administered mu- and delta-, but not kappa-, opioid receptor agonists. J Pharmacol Exp Ther 2002;302:1146-50). This model was utilized to evaluate compound A for its ability to inhibit allodynia.

Allodynia was induced as described by Sluka, et al. (Sluka K, Kalra A, Moore S. Unilateral intramuscular injections of acidic saline produce a bilateral, long-lasting hyperalgesia. Muscle Nerve 2001;24:37-46) with minor modifications. On Day 0, male Sprague-Dawley rats (˜200 g body weight) in their dark cycle were placed in suspended wire-bottom cages and allowed to acclimate for 0.5 hours. The baseline paw withdrawal threshold was determined on the right hind paw by Von Frey monofilament hairs (bending forces of 2.0, 3.6, 5.5, 8.5, 15.1, and 28.8 g) using the Dixon Up-Down method (Dixon W. Efficient analysis of experimental observations. Ann Rev Pharmacol Toxicol 1980;20:441-62). Von Frey hairs were applied to the plantar surface for up to 6 seconds, and a flinching of the paw during that time frame was considered a positive response. After assessment, the right gastrocnemius muscle was shaved, swabbed with alcohol, and injected with 0.1 mL of 0.9% NaCl solution acidified to pH 4 with HCl. The injection was repeated on Day 5. Animals were manipulated with a dynamic plantar aesthesiometer (Ugo Basile, Comerio-Varese, Italy) on Days 6, 7, and 8 to facilitate induction of the allodynia. To screen the rats for the development of allodynia, the 15.1 g Von Frey hair was applied to the ipsilateral paw on Day 11. Positive responders from that test were included in the compound evaluation study. On Day 12 (the day of peak allodynia), animals were assigned into treatment groups, and then their ipsilateral paw withdrawal thresholds were determined to establish the allodynia (reduction in paw withdrawal threshold) compared to baseline values. Rats were then orally dosed with 10 mL/kg vehicle (0.5% hydroxypropyl-methylcellulose/0.2% Tween 80) or the indicated dose of compound A. Paw withdrawal thresholds were reassessed by Von Frey hairs in blinded fashion 2 hours after dosing for the dose-response study, and 2, 5, 8, and 24 hours after dosing in the time course experiment. The inhibition of allodynia was determined for each animal by dividing the increase in paw withdrawal threshold after treatment by the difference between baseline and pretreatment paw withdrawal values. This fraction was then converted to percent inhibition by multiplying by 100.

Compound A dose-dependently attenuated allodynia, with a minimum effective doses of 10 mg/kg (Table I). To determine the time course of inhibition, allodynia was monitored at various time points after a 10 mg/kg dose of compound A. Administration of compound A significantly reversed PWT at each time point after oral dosing; however, it was most effective from 2 to 5 hours after dosing (Table II).

TABLE I
Paw Withdrawal Thresholds in Rats Before and After
Acidic Saline Injection, Comparison of Oral Treatment
with Vehicle and Compound A

| | Mean Paw | Mean Paw | |
|---|---|---|---|
| | Withdrawal | Withdrawal | |
| | Threshold before | Threshold 2 hr | Mean % |
| | dose | after dose | Inhibition of |
| Treatment | (g ± S.E.M.) | (g ± S.E.M.) | Allodynia |
| Baseline (before | 21.9 ± 1.5 | Not applicable | |
| acid injection or | | | |
| drug treatment) | | | |
| N = 24 | | | |
| Vehicle | 6.8 ± 1.0 | 6.4 ± 1.1 | 0% |
| N = 8 | | | |
| 3 mg/kg | 6.3 ± 0.8 | 11.4 ± 1.7 | 33% |
| Compound A (N = 8) | | | |
| 10 mg/kg | 6.2 ± 1.0 | 17.4 ± 1.5 | 72% |
| Compound A | | | |
| (N = 8) | | | |

TABLE II
Paw Withdrawal Thresholds in Rats After Acidic Salin
Injection, Comparison of Oral Treatment with Vehicle or
Compound A at Different Times After Dosing

| | | | Mean Paw | |
|---|---|---|---|---|
| | | Baseline | Withdrawal | |
| | | Mean Paw | Threshold | Mean % |
| | Time After | Withdrawal | after | Inhibition |
| | Treatment | Threshold | treatment | of |
| Treatment | (hours) | (g ± S.E.M.) | (g ± S.E.M.) | Allodynia |
| Baseline | | 22.0 ± 1.1 | Not applicable | |
| (before acid | | | | |
| injection or | | | | |
| drug | | | | |
| treatment) | | | | |
| N = 10 | | | | |
| Vehicle | Prior to | | 6.3 ± 0.8 | |
| (N = 7) | Treatment | | | |
| | 2 | | 7.0 ± 1.3 | 0% |
| | 5 | | 4.2 ± 0.6 | 0% |
| | 8 | | 4.5 ± 0.6 | 0% |
| | 24 | | 5.9 ± 1.1 | 0% |
| 10 mg/kg | Prior to | | 6.5 ± 0.9 | |
| Compound A | Treatment | | | |
| (N = 8) | 2 | | 16.0 ± 1.4 | 60% ± 10 |
| | 5 | | 15.2 ± 1.8 | 62% ± 10 |
| | 8 | | 9.9 ± 2.0 | 31% ± 12 |
| | 24 | | 13.3 ± 2.8 | 46% ± 17 |

Thus, administration of compound A reduced tactile allodynia to the footpad caused by prior injection of acidic saline. Efficacy was sustained throughout a 24 hour observation period after dosing, although efficacy declined slightly over time.

The results indicate that compound A is useful for treating the allodynia associated with fibromyalgia syndrome.

### EXAMPLES

The following examples are illustrative of the instant invention; they are not intended to limit the scope.

In Examples 1 to 8, the first step involves the conversion of a cyclic ketone to an α,β-unsaturated ester 2 via use of a trialkylphosphonoacetate or an (alkoxycarbonylmethyl)triphenylphosphonium halide and a base, such as sodium hydride, potassium hydride, lithium- or sodium- or potassium-hexamethyldisilazide, butyllithium or potassium t-butoxide in a solvent such as tetrahydrofuran, dimethylformamide, diethylether or dimethylsulfoxide at a suitable temperature in the range from −78° C. to 100° C.

The second step involves reaction of the α,β-unsaturated ester 2 with nitromethane and a suitable base such as tetrabutylammonium fluoride, tetramethylguanidine, 1,5-diazabicyclo[4,3,0]non-5-ene, 1,8-diazabicyclo[5,4,0]undec-7-ene, a sodium or potassium alkoxide, sodium hydride or potassium fluoride in a solvent such as tetrahydrofuran, diethylether, dimethylformamide, dimethylsulphoxide, benzene, toluene, dichloromethane, chloroform or tetrachloromethane at a suitable temperature in the range from −20° C. to 100° C.

The third step involves catalytic hydrogenation of the nitro moiety of 3 using a catalyst such as Raney nickel, palladium on charcoal or rhodium catalyst or other nickel or palladium containing catalyst in a solvent such as methanol, ethanol, isopropanol, ethyl acetate, acetic acid, 1,4-dioxane, chloroform or diethyl ether at a suitable temperature in the range from 20° C. to 80° C.

The fourth step involves hydrolysis of lactam 4 using hydrochloric acid and may also utilize a co-solvent such tetrahydrofuran or 1,4-dioxane or other such inert water miscible solvent at a suitable temperature in the range from 20° C. to reflux.

### EXAMPLE 1

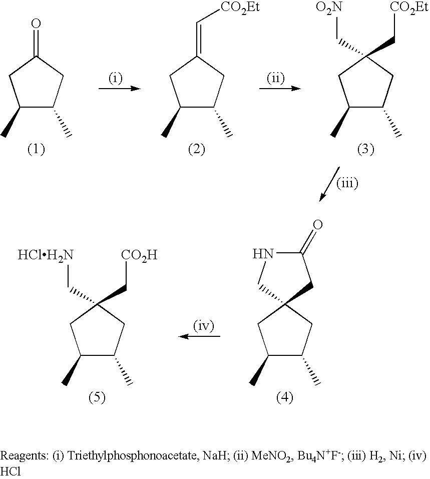

Synthesis of (trans)-(3,4-Dimethyl-cyclopentylidene)-acetic Acid Ethyl Ester (2)

NaH (60% dispersion in oil, 737 mg, 18.42 mmol) was suspended in dry tetrahydrofuran (50 mL) and cooled to 0° C. Triethylphosphonoacetate (3.83 mL, 19.30 mmol) was added and the mixture stirred at 0° C. for 15 minutes. The ketone (1) (1.965 g, 17.54 mmol) in THF (10 mL) was then added and the mixture allowed to warm to room temperature. After 2 hours, the mixture was partitioned between diethyl ether (200 mL) and water (150 mL). The organic phase was separated, washed with brine, dried (MgSO₄) and the solvent removed in vacuo. The residue was purified by flash chromatography (silica, ethyl acetate:heptane 1:9) to give 3.01 g (94%) of (2) as a colorless oil. ¹H NMR 400 MHz (CDCl₃): δ 1.01 (3H, d, J=6 Hz), 1.03 (3H, d, J=6 Hz), 1.26 (3H, t, J=7 Hz), 1.49 (2H, m), 2.07 (1H, m), 2.24 (1H, m), 2.61 (1H, m), 4.13 (2H, q, J=7 Hz), 5.72 (1H, s).

MS (CI+) m/e: 183 ([MH⁺], 18%).

Synthesis of (trans)-(3,4-Dimethyl-1-nitromethyl-cyclopentyl)-acetic Acid Ethyl Ester (3) The unsaturated ester (2) (2.95 g, 16.2 mmol) was dissolved in tetrahydrofuran (10 mL) and stirred at 70° C. with nitromethane (1.9 mL, 35.2 mmol) and tetrabutylammonium fluoride (1.0 M in tetrahydrofuran, 22 mL, 22.0 mmol). After 6 hours, the mixture was cooled to room temperature, diluted with ethyl acetate (50 mL), and washed with 2N HCl (30 mL) followed by brine (50 mL). The organic phase was collected, dried (MgSO₄) and the solvent removed in vacuo. The residue was purified by flash chromatography (silica, ethyl acetate:heptane 1:9) to give 1.152 g (29%) of a clear oil.

¹H NMR 400 MHz (CDCl₃): δ 0.98 (6H, d, J=6 Hz), 1.10-1.39 (5H, m), 1.47 (2H, m), 1.87 (1H, m), 2.03 (1H, m), 2.57 (2H, ABq, J=16, 38 Hz), 4.14 (2H, q, J=7 Hz), 4.61 (2H, ABq, J=12, 60 Hz).

MS (ES+) m/e: 244 ([MH⁺], 8%).

IR (film) ν cm⁻¹: 1186, 1376, 1549, 1732, 2956.

Synthesis of (±)-(trans)-7,8-Dimethyl-2-aza-spiro[4.4]nonan-2-one (4)

The nitroester (3) (1.14 g, 4.7 mmol) was dissolved in methanol (50 mL) and shaken over Raney nickel catalyst under an atmosphere of hydrogen (40 psi) at 30° C. After 5 hours, the catalyst was removed by filtration through celite. The solvent was removed in vacuo to give 746 mg (95%) of a pale yellow oil which solidified on standing.

¹H NMR 400 MHz (CDCl₃): δ 0.98 (6H, d, J=6 Hz), 1.32 (2H, mn), 1.46 (2H, m), 1.97 (2H, m), 2.27 (2H, ABq, J=16, 27 Hz), 3.23 (2H, s), 5.62 (1H, br s).

MS (ES+) m/e: 168 ([MH⁺], 100%).

IR (film) ν cm⁻¹: 1451, 1681, 1715, 2948, 3196.

Synthesis of (±)-(trans)-(1-Aminomethyl-3,4-dimethyl-cyclopentyl)-acetic Acid Hydrochloride (5)

The lactam (4) (734 mg, 4.40 mmol) was heated to reflux in a mixture of 1,4-dioxan (5 mL) and 6N HCl (15 mL). After 4 hours, the mixture was cooled to room temperature, diluted with water (20 mL), and washed with dichloromethane (3×30 mL). The aqueous phase was collected and the solvent removed in vacuo. The residue was triturated with ethyl acetate to give 675 mg (69%) of a white solid after collection and drying.

¹H NMR 400 MHz (d₆-DMSO): δ 0.91 (6H, d, J=6 Hz), 1.18 (2H, m), 1.42 (2H, m), 1.72 (1H, m), 1.87 (1H, m), 2.42 (2H, ABq, J=16, 24 Hz), 2.90 (2H, ABq, J=12, 34 Hz), 8.00 (3H, br s), 12.34 (1H, br s).

MS (ES+) m/e: 186 ([MH-HCl]⁺, 100%).

### EXAMPLE 2

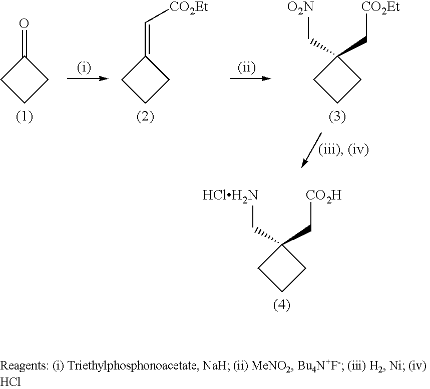

Synthesis of Cyclobutylidene-acetic Acid Ethyl Ester (2)

NaH (60% dispersion in oil, 1.80 g, 44.94 mmol) was suspended in dry tetrahydrofuran (80 mL) and cooled to 0° C. Triethylphosphonoacetate (9.33 mL, 47.08 mmol) was added and the mixture stirred at 0° C. for 15 minutes. Cyclobutanone (1) (3.0 g, 42.8 mmol) in THF (20 mL) was then added and the mixture allowed to warm to room temperature. After 2 hours, the mixture was partitioned between diethyl ether (200 mL) and water (150 mL). The organic phase was separated, washed with brine, dried (MgSO₄), and the solvent removed in vacuo at 600 mm Hg. The residue was purified by flash chromatography (silica, ethyl acetate:pentane 1:19) to give 5.81 g (96%) of (2) as a colorless oil.

¹H NMR, 400 MHz (CDCl₃): δ 1.27 (3H, t, J=6 Hz), 2.09 (2H, m), 2.82 (2H, m), 3.15 (2H, m), 4.14 (2H, q, J=6 Hz), 5.58 (1H, s).

MS (ES+) m/e: 141 ([MH⁺], 100%). IR (film) ν cm⁻¹: 1088, 1189, 1336, 1673, 1716, 2926.

Synthesis of (1-Nitromethyl-cyclobutyl)-acetic Acid Ethyl Ester (3)

The unsaturated ester (2) (5.79 g, 41.4 mmol) was dissolved in tetrahydrofuran (20 mL) and stirred at 70° C. with nitromethane (4.67 mL, 86.4 mmol) and tetrabutylammonium fluoride (1.0 M in tetrahydrofuran, 55 mL, 55.0 mmol). After 18 hours, the mixture was cooled to room temperature, diluted with ethyl acetate (150 mL), and washed with 2N HCl (60 mL) followed by brine (100 mL). The organic phase was collected, dried (MgSO₄), and the solvent removed in vacuo. The residue was purified by flash chromatography (silica, ethyl acetate:heptane 1:1) to give 4.34 g (52%) of a clear oil.

¹H NMR 400 MHz (CDCl₃): δ 1.27 (3H, t, J=6 Hz), 1.96-2.20 (6H, m), 2.71 (2H, s), 4.15 (2H, q, J=6 Hz), 4.71 (2H, s).

MS (ES+) m/e: 202 ([MH⁺], 100%).

IR (film) ν cm⁻¹: 1189, 1378, 1549, 1732, 2984.

Synthesis of (1-Aminomethyl-cyclobutyl)-acetic Acid Hydrochloride (4)

The nitroester (3) (2.095 g, 10.4 mmol) was dissolved in methanol (50 mL) and shaken over Raney nickel catalyst under an atmosphere of hydrogen (45 psi) at 30° C. After 6 hours, the catalyst was removed by filtration through celite. The solvent was removed in vacuo to give 1.53 g of a pale yellow oil which was used without purification. The oil was dissolved in 1,4-dioxane (5 mL) and 6N HCl (15 mL) and heated to reflux. After 5 hours, the mixture was cooled to room temperature, diluted with water (20 mL), and washed with dichloromethane (3×30 mL). The aqueous phase was collected and the solvent removed in vacuo.

The residue was triturated with ethyl acetate to give 1.35 g (72%) of a white solid after collection and drying.

¹H NMR 400 MHz (d6-DMSO): δ 1.80-2.03 (6H, m), 2.59 (2H, s), 3.02 (2H, s), 8.04 (3H, br s), 12.28 (1H, br s).

MS (ES+) m/e: 144 ([MH-HCl]⁺, 100%).

Microanalysis calculated for C₇H₁₄NO₂Cl:

C, 46.80%; H, 7.86%; N, 7.80%.

Found: C, 46.45%; H, 7.98%; N, 7.71%.

### EXAMPLE 3

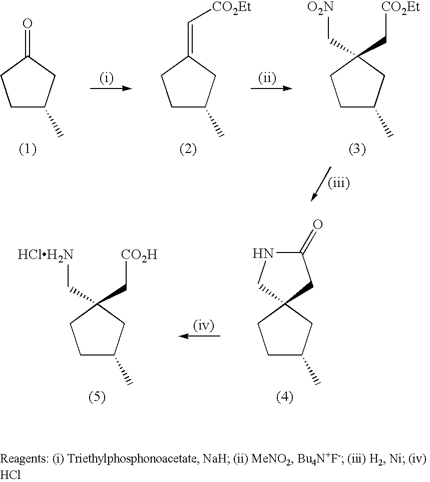

Synthesis of (R)-(3-Methyl-cyclopentylidene)-acetic Acid Ethyl Ester (2)

NaH (60% dispersion in oil, 1.86 g, 46.5 mmol) was suspended in dry tetrahydrofuran (40 mL) and cooled to 0° C. Triethylphosphonoacetate (9.69 mL, 48.8 mmol) was added and the mixture stirred at 0° C. for 15 minutes. The ketone (1) (5 ml, 46.5 mmol) in THF (10 mL) was then added and the mixture allowed to warm to room temperature. After 2 hours, the mixture was partitioned between diethyl ether (200 mL) and water (150 mL). The organic phase was separated, washed with brine, dried (MgSO₄) and the solvent removed in vacuo. The residue was purified by flash chromatography (silica, ethyl acetate:heptane 1:9) to give 5.45 g (70%) of (2) as a colorless oil.

¹H NMR 400 MHz (CDCl₃): δ 1.04 (3H, m), 1.27 (3H, t, J=7 Hz), 1.80-2.74 (7H, m), 2.90-3.15 (1H, m), 4.13 (2H, q, J=7 Hz), 5.76 (1H, s).

MS (CI+) m/e: 169 ([MH⁺], 20%).

IR (film) ν cm⁻¹: 1205, 1371, 1653, 1716, 2955.

Synthesis of (cis/trans)-(3R)-(3-Methyl-1-nitromethyl-cyclopentyl)-acetic Acid Ethyl Ester (3)

The unsaturated ester (2) (3.0 g, 17.8 mmol) was dissolved in tetrahydrofuran (20 mL) and stirred at 70° C. with nitromethane (1.92 mL, 35.6 mmol) and tetrabutylammonium fluoride (1.0 M in tetrahydrofuran, 25 mL, 25.0 mmol). After 18 hours, the mixture was cooled to room temperature, diluted with ethyl acetate (50 mL), and washed with 2N HCl (30 mL) followed by brine (50 mL). The organic phase was collected, dried (MgSO₄), and the solvent removed in vacuo. The residue was purified by flash chromatography (silica, ethyl acetate:heptane 1:9) to give 2.00 g (49%) of a clear oil.

¹H NMR 400 MHz (CDCl₃): δ 1.02 (3H, d, J=6 Hz), 1.08-1.37 (5H, m), 1.59-2.17 (5H, m), 2.64 (2H, m), 4.15 (2H, q, J=7 Hz), 4.64 (2H, m).

MS (ES+) m/e: 230 ([MH⁺], 4%).

IR (film) ν cm⁻¹: 1183, 1377, 1548, 1732, 2956.

Synthesis of (cis/trans)-(7R)-7-Methyl-2-aza-spiro[4.4]nonan-2-one (4)

The nitroester (3) (1.98 g, 8.66 mmol) was dissolved in methanol (50 mL) and shaken over Raney nickel catalyst under an atmosphere of hydrogen (40 psi) at 30° C. After 18 hours, the catalyst was removed by filtration through celite. The solvent was removed in vacuo and the residue purified by flash chromatography (silica, ethyl acetate:heptane 1:1) to give 1.05 g (79%) of a white solid.

¹H NMR 400 MHz (CDCl₃): δ 1.03 (3H, m), 1.22 (2H, m), 1.60-2.15 (5H, m), 2.22 (2H, m), 3.20 and 3.27 (2H total, 2×s, cis, and trans), 6.18 (1H, br s).

MS (ES+) m/e: 154 ([MH⁺], 100%).

IR (film) ν cm⁻¹: 1695, 2949, 3231.

Synthesis of (cis/trans)-(3R)-(1-Aminomethyl-3-methyl-cyclopentyl)-acetic Acid Hydrochloride (5)

The lactam (4) (746 mg, 4.88 mmol) was heated to reflux in a mixture of 1,4-dioxan (5 mL) and 6N HCl (15 mL). After 4 hours, the mixture was cooled to room temperature, diluted with water (20 mL), and washed with dichloromethane (3×30 mL). The aqueous phase was collected and the solvent removed in vacuo. The residue was triturated with ethyl acetate to give a white solid which was collected and dried. This was recrystallized from ethyl acetate/methanol to give 656 mg (65%) of (5) after collection and drying.

¹H NMR 400 MHz (d6-DMSO): δ 0.96 (3H, m), 1.01-1.24 (2H, m), 1.42-2.10 (5H, m), 2.41 and 2.44 (2H total, 2×s, cis/trans), 2.94 (2H, m), 7.96 (3H, br s), 12.35 (1H, br s).

MS (ES+) m/e: 172 ([MH-HCl]⁺, 100%).

### EXAMPLE 4

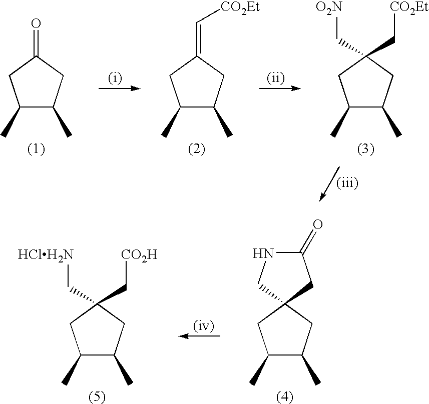

Reagents: (i) Triethylphosphonoacetate, NaH; (ii) MeNO₂, Bu₄N⁺F⁻; (iii) H₂, Ni; (iv) HCl

Synthesis of (cis)-(3,4-Dimethyl-cyclopentylidene)-acetic Acid Ethyl Ester (2)

NaH (60% dispersion in oil, 519 mg, 12.96 mmol) was suspended in dry tetrahydrofuran (30 mL) and cooled to 0° C. Triethylphosphonoacetate (2.68 mL, 13.5 mmol) was added and the mixture stirred at 0° C. for 15 minutes. The ketone (1) (1.21 g, 10.80 mmol) in THF (10 mL) was then added and the mixture allowed to warm to room temperature. After 2 hours, the mixture was partitioned between diethyl ether (200 mL) and water (150 mL). The organic phase was separated, washed with brine, dried (MgSO₄) and the solvent removed in vacuo. The residue was purified by flash chromatography (silica, ethyl acetate:heptane 5:95) to give 1.40 g (71%) of (2) as a colorless oil.

¹H NMR 400 MHz (CDCl 13): δ 0.84 (3H, d, J=6 Hz), 0.91 (3H, d, J=6 Hz), 1.26 (3H, t, J=7 Hz), 2.01-2.95 (6H, m), 4.13 (2H, q, J=7 Hz), 5.76 (1H, s).

MS (CI+) m/e: 183 ([MH⁺], 18%).

IR (film) ν cm⁻¹: 1043, 1125, 1200, 1658, 1715, 2959.

Synthesis of (trans)-(3,4-Dimethyl-1-nitromethyl-cyclopentyl)-acetic Acid Ethyl Ester (3)

The unsaturated ester (2) (1.384 g, 7.60 mmol) was dissolved in tetrahydrofuran (10 mL) and stirred at 70° C. with nitromethane (0.82 mL, 15.2 mmol) and tetrabutylammonium fluoride (1.0M in tetrahydrofuran, 11.4 mL, 11.4 mmol). After 6 hours, the mixture was cooled to room temperature, diluted with ethyl acetate (50 mL) and washed with 2N HCl (30 mL) followed by brine (50 mL). The organic phase was collected, dried (MgSO₄), and the solvent removed in vacuo. The residue was purified by flash chromatography (silica, ethyl acetate:heptane 5:95) to give 0.837 g (45%) of a clear oil.

¹H NMR 400 MHz (CDCl₃): δ 0.91 (6H, d, J=6 Hz), 1.21-1.39 (5H, m), 1.98 (2H, m), 2.18 (2H, m), 2.64 (2H, s), 4.15 (2H, q, J=7 Hz), 4.61 (2H, s).

MS (ES+) m/e: 244 ([MH⁺], 8%).

IR (film) ν cm⁻¹: 1184, 1377, 1548, 1732, 2961.

Synthesis of (trans)-7,8-Dimethyl-2-aza-spiro[4.4]nonan-2-one (4)

The nitroester (3) (0.83 g, 3.4 mmol) was dissolved in methanol (30 mL) and shaken over Raney nickel catalyst under an atmosphere of hydrogen (40 psi) at 30° C. After 4 hours, the catalyst was removed by filtration through celite. The solvent was removed in vacuo to give 567 mg (99%) of a pale yellow oil which solidified on standing.

¹H NMR 400 MHz (CDCl₃): δ 0.89 (6H, d, J=6 Hz), 1.38 (2H, m), 1.91 (2H, m), 2.10 (2H, m), 2.32 (2H, s), 3.18 (2H, s), 5.61 (1H, br s).

MS (ES+) m/e: 168 ([MH⁺], 100%).

IR (film) ν cm⁻¹: 1304, 1450, 1699, 2871, 3186.

Synthesis of (1α,3β,4β)-(1-Aminomethyl-3,4-dimethyl-cyclopentyl)-acetic Acid Hydrochloride (5)

The lactam (4) (563 mg, 4.40 mmol) was heated to reflux in a mixture of 1,4-dioxan (5 mL) and 6N HCl (15 mL). After 4 hours, the mixture was cooled to room temperature, diluted with water (20 mL), and washed with dichloromethane (3×30 mL). The aqueous phase was collected and the solvent removed in vacuo. The residue was triturated with ethyl acetate to give a white solid which was collected and dried. This was recrystallized from ethyl acetate/methanol to give 440 mg (59%) of (5) after collection and drying.

¹H NMR 400 MHz (d₆-DMSO): δ 0.84 (6H, d, J=6 Hz), 1.21 (2H, m), 1.81 (2H, m), 2.06 (2H, m), 2.47 (2H, s), 2.89 (2H, s), 7.94 (3H, br s), 12.30 (1H, br s).

MS (ES+) m/e: 186 ([MH-HCl]⁺, 100%).

### EXAMPLE 5

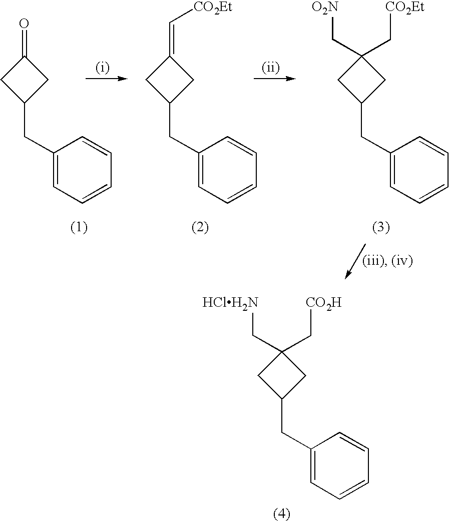

Reagents: (i) Triethylphosphonoacetate, NaH; (ii) MeNO₂, Bu₄N⁺F⁻; (iii) H₂, Ni; (iv) HCl

Synthesis of (3-Benzyl-cyclobutylidene)-acetic Acid Ethyl Ester (2)

NaH (60% dispersion in oil, 0.496 g, 12.4 mmol), was suspended in dry tetrahydrofuran (40 mL) and cooled to 0° C. Triethylphosphonoacetate (2.58 mL, 13.0 mmol) was added and the mixture stirred at 0° C. for 15 minutes. The cyclobutanone (1) (1.89 g, 11.8 mmol) in THF (15 mL) was then added and the mixture allowed to warm to room temperature. After 4 hours, the mixture was partitioned between diethyl ether (200 mL) and water (150 mL). The organic phase was separated, washed with brine, dried (MgSO₄), and the solvent removed in vacuo. The residue was purified by flash chromatography (silica, ethyl acetate:heptane 1:4) to give 2.19 g (81%) of (2) as a colorless oil.

¹H NMR 400 MHz (CDCl₃): δ 1.26 (3H, t, J=6 Hz), 2.55 (1H, m), 2.64-2.95 (5H, m), 3.28 (2H, m), 4.14 (2H, q, J=6 Hz), 5.63 (1H, s), 7.10-7.32 (5H, m).

MS (ES+) m/e: 231 ([MH⁺], 8%).

IR (film) ν cm⁻¹: 1190, 1335, 1675, 1715, 2980.

Synthesis of (cis/trans)-(3-Benzyl-1-nitromethyl-cyclobutyl)-acetic Acid Ethyl Ester (3)

The unsaturated ester (2) (2.17 g, 9.42 mmol) was dissolved in tetrahydrofuran (15 mL) and stirred at 70° C. with nitromethane (1.02 mL, 18.8 mmol) and tetrabutylammonium fluoride (1.0 M in tetrahydrofuran, 14 mL, 14.0 mmol). After 24 hours, the mixture was cooled to room temperature, diluted with ethyl acetate (150 mL), and washed with 2N HCl (60 mL) followed by brine (100 mL). The organic phase was collected, dried (MgSO₄) and the solvent removed in vacuo. The residue was purified by flash chromatography (silica, ethyl acetate:heptane 1:1) to give 1.55 g (57%) of a clear oil.

¹H NMR 400 MHz (CDCl₃): δ 1.25 (3H, m), 1.86 (2H, m), 2.09-2.33 (2H, m), 2.53-2.78 (3H, m), 4.15 (2H, q, J=6 Hz), 4.62 and 4.71 (2H total, 2×s, cis/trans), 7.08-7.34 (5H, m).

MS (ES+) m/e: 292 ([MH⁺], 100%).

IR (film) ν cm⁻¹: 1185, 1378, 1549, 1732, 2933.

Synthesis of (cis/trans)-(1-Aminomethyl-3-benzyl-cyclobutyl)-acetic Acid Hydrochloride (4)

The nitroester (3) (1.53 g, 5.25 mmol) was dissolved in methanol (50 mL) and shaken over Raney nickel catalyst under an atmosphere of hydrogen (45 psi) at 30° C. After 5 hours, the catalyst was removed by filtration through celite. The solvent was removed in vacuo to give 1.32 g of a pale yellow oil which was used without purification. The oil was dissolved in 1,4-dioxane (5 mL) and 6N HCl (15 mL) and heated to reflux. After 4 hours, the mixture was cooled to room temperature, diluted with water (20 mL) and washed with dichloromethane (3×30 mL). The aqueous phase was collected and the solvent removed in vacuo. The residue was triturated with ethyl acetate to give 0.88 g (62%) of a white solid after collection and drying.

¹H NMR 400 MHz (d6-DMSO): δ 1.64 (1H, m), 1.84 (2H, m), 2.07 (1H, m), 2.20-2.74 (5H, m), 2.98 and 3.04 (2H total, 2×s, cis/trans), 7.10-7.31 (5H, m), 8.00 (3H, br s), 12.28 (1H, br s).

MS (ES+) m/e: 234 ([MH-HCl]⁺, 100%).

### EXAMPLE 6

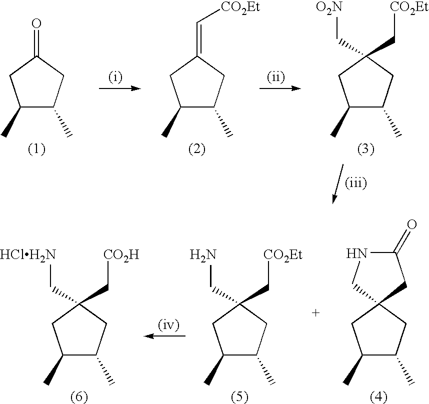

Reagents: (i) Triethylphosphonoacetate, NaH; (ii) MeNO₂, Bu₄N⁺F⁻; (iii) H₂, Ni; (iv) Hcl.

Ketone (1) is known in the literature and can be synthesized by the methods outlined therein: Y. Kato, Chem. Pharm. Bull., 1966;14:1438-1439 and related references: W. C. M. C. Kokke, F. A. Varkevisser, J. Org. Chem., 1974;39:1535; R. Baker, D. C. Billington, N. Eranayake, JCS Chem. Comm., 1981:1234; K. Furuta, K. Iwanaga, H. Yamamoto, Tet. Lett., 1986;27:4507; G. Solladie, O. Lohse, Tet. Asymm., 1993;4:1547; A. Rosenquist, I. Kvarnstrom, S. C. T. Svensson, B. Classon, B. Samuelsson, Acta Chem. Scand., 1992;46:1127; E. J. Corey, W. Su, Tet. Lett., 1988;29:3423; D. W. Knight, B. Ojhara, Tet. Lett., 1981;22:5101.

Synthesis of (trans)-(3,4-Dimethyl-cyclopentylidene)-acetic Acid Ethyl Ester (2)

To a suspension of sodium hydride (1.3 g, 32.5 mmol) in THF (60 mL) under nitrogen at 0° C. was added triethylphosphonoacetate (6.5 mL, 32.7 mmol) over 5 minutes. After stirring for a further 10 minutes, a solution of (1) (approx. 2.68 g, approx. 30 mmol) in THF (2×10 mL) was added to the now clear solution and the ice bath removed. After 4 hours the reaction was quenched by pouring into water (100 mL) and the mixture extracted with ether (400 mL). The organic phase was washed with saturated brine (100 mL), dried and concentrated in vacuo. Column chromatography (10:1 heptane/ethyl acetate) gave the product as an oil, 4.53 g, approx. 100%; 91%.

¹H NMR 400 MHz (CDCl₃): δ 1.01 (3H, d, J=6 Hz), 1.03 (3H, d, J=6 Hz), 1.26 (3H, t, J=7 Hz), 1.49 (2H, m), 2.07 (1H, m), 2.24 (1H, m), 2.61 (1H, m), 4.13 (2H, q, J=7 Hz), 5.72 (1H, s).

MS (CI+) m/e: 183 ([MH⁺], 21%).

Synthesis of (trans)-(3,4-Dimethyl-1-nitromethyl-cyclopentyl)-acetic Acid Ethyl Ester (3)

To a solution of (2) (4.24 g, 23.3 mmol) in THF (15 mL) was added TBAF (32 mL of a 1 M solution in THF, 32 mmol) followed by nitromethane (3 mL) and the reaction heated at 60° C. for 8 hours. After cooling, the reaction mixture was diluted with ethyl acetate (150 mL) and washed with 2N HCl (40 mL) then saturated brine (50 mL). Column chromatography (10:1 heptane/ethyl acetate) gave the product as an oil, 2.24 g, 40%.

¹H NMR 400 MHz (CDCl₃): δ 0.98 (6H, d, J=6 Hz), 1.10-1.39 (5H, m), 1.47 (2H, m), 1.87 (1H, m), 2.03 (1H, m), 2.57 (2H, ABq, J=16, 38 Hz), 4.14 (2H, q, J=7 Hz), 4.61 (2H, ABq, J=12, 60 Hz).

MS (ES+) m/e: 244 ([MH⁺], 5%).

IR (film) ν cm⁻¹: 1186, 1376, 1549, 1732, 2956.

Synthesis of (3S,4S)-(1-Aminomethyl-3,4-dimethyl-cyclopentyl)-acetic Acid Hydrochloride (6)

A solution of (3) (3.5 g, 14.4 mmol) in methanol (100 mL) in the presence of Ni sponge was hydrogenated at 30° C. and 50 psi for 4 hours. Filtering off the catalyst and concentrating in vacuo gave a 2:1 mixture of lactam and aminoester, 2.53 g, calculated as 96%, which was used without purification. This mixture (2.53 g, 13.8 mmol) in dioxane (15 mL) and 6N HCl (45 mL) was heated under reflux (oil bath=110° C.) for 4 hours. After cooling and diluting with water (60 mL), the mixture was washed with dichloromethane (3×50 mL) and then concentrated in vacuo. The resulting oil was washed with ethyl acetate then dichloromethane to give a sticky foam which was dried to give the product as a white powder, 2.32 g, 76%.

α_{D} (23° C.) (H₂O) (c=1.002)=+28.2°.

¹H NMR 400 MHz (d₆-DMSO): δ 0.91 (6H, d, J=6 Hz), 1.18 (2H, m), 1.42 (2H, m), 1.72 (1H, m), 1.87 (1H, m), 2.42 (2H, ABq, J=16, 24 Hz), 2.90 (2H, ABq, J=12, 34 Hz), 8.00 (3H, br s), 12.34 (1H, br s).

MS (ES+) m/e: 186 ([MH-HCl]⁺, 100%).

### EXAMPLE 7

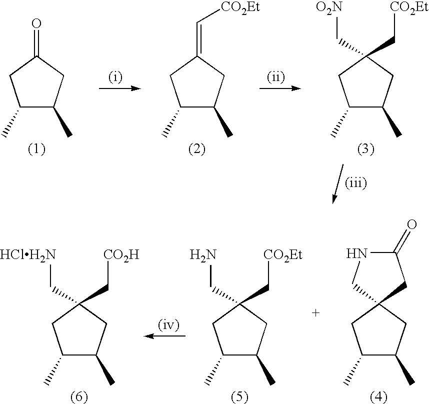

Ketone (1) is known in the literature and can be synthesized by the methods outlined therein: W. C. M. C. Kokke, F. A. Varkevisser, J. Org. Chem., 1974;39 1535; Cammalm, Ark. Kemi, 1960;15:215, 219; Cammalm, Chem. Ind., 1956:1093; Linder et al., J. Am. Chem. Soc., 1977;99:727, 733; A. E. Greene, F. Charbonnier, Tet. Lett., 1985;26:5525 and related references: R. Baker, D. C. Billington, N. Eranayake, JCS Chem. Comm., 1981:1234; K. Furuta, K. Iwanaga, H. Yamamoto, Tet. Lett., 1986;27:4507; G. Solladie, O. Lohse, Tet. Asymm., 1993;4:1547; A. Rosenquist, I. Kvarnstrom, S. C. T. Svensson, B. Classon, B. Samuelsson, Acta Chem. Scand., 1992;46:1127; E. J. Corey, W. Su, Tet. Lett., 1988;29:3423; D. W. Knight, B. Ojhara. Tet. Lett., 1981;22:5101.

Synthesis of (trans)-(3,4-Dimethyl-cyclopentylidene)-acetic Acid Ethyl Ester (2)

To a suspension of sodium hydride (0.824 g, 20.6 mmol) in THF (40 mL) under nitrogen at 0° C. was added triethylphosphonoacetate (4.1 mL, 20.7 mmol) over 5 minutes. After stirring for a further 10 minutes, a solution of (1) (approx. 2.10 g, approx. 15.8 mmol) in THF (2×10 mL) was added to the now clear solution and the ice bath removed. After 4 hours, the reaction was quenched by pouring into water (100 mL) and the mixture extracted with ether (4×100 mL). The organic phase was washed with saturated brine (50 mL), dried and concentrated in vacuo. Column chromatography (10:1 heptane/ethyl acetate) gave the product as an oil, 2.643 g, approx. 100%; 91%.

¹H NMR 400 MHz (CDCl₃): δ 1.01 (3H, d, J=6 Hz), 1.03 (3H, d, J=6 Hz), 1.26 (3H, t, J=7 Hz), 1.49 (2H, m), 2.07 (1H, m), 2.24 (1H, m), 2.61 (1H, m), 4.13 (2H, q, J=7 Hz), 5.72 (1H, s).

MS (CI+) m/e: 183 ([MH⁺], 19%).

Synthesis of (trans)-(3,4-Dimethyl-1-nitromethyl-cyclopentyl)-acetic Acid Ethyl Ester (3)

To a solution of (2) (2.44 g, 13.4 mmol) in THF (12 mL) was added TBAF (18 mL of a 1 M solution in THF, 18 mmol) followed by nitromethane (2 mL) and the reaction heated at 60° C. for 4 hours. After cooling, the reaction mixture was diluted with ethyl acetate (250 mL) and washed with 2N HCl (50 mL) then saturated brine (50 mL). Column chromatography (10:1 heptane/ethyl acetate) gave the product as an oil, 1.351g, 41%.

¹H NMR 400 MHz (CDCl₃): δ 0.98 (6H, d, J=6 Hz), 1.10-1.39 (5H, m), 1.47 (2H, m), 1.87 (1H, m), 2.03 (1H, m), 2.57 (2H, ABq, J=16, 38 Hz), 4.14 (2H, q, J=7 Hz), 4.61 (2H, ABq, J=12,60 Hz).

MS (ES+) m/e: 244 ([MH⁺], 12%).

IR (film) ν cm⁻¹: 1186, 1376, 1549, 1732, 2956.

Synthesis of (3R,4R)-(1-Aminomethyl-3,4-dimethyl-cyclopentyl)-acetic Acid Hydrochloride (6)

A solution of (3) (1.217 g, 5.0 mmol) in methanol (100 mL) in the presence of Ni sponge was hydrogenated at 30° C. and 50 psi for 4 hours. Filtering off the catalyst and concentrating in vacuo gave a 3:5 mixture of lactam and aminoester, 1.00 g, calculated as 100%, which was used without purification. This mixture (1.00 g, 5.0 mmol) in dioxane (10 mL) and 6N HCl (30 mL) was heated under reflux (oil bath=110° C.) for 4 hours. After cooling and diluting with water (100 mL), the mixture was washed with dichloromethane (2×50 mL) and then concentrated in vacuo. The resulting oil was washed with ethyl acetate then dichloromethane to give a sticky foam which was dried to give the product as a white powder, 0.532 g, 48%.

α_{D} (23° C.) (H₂O) (c=1.01)=−27.0°.

¹H NMR 400 MHz (d₆-DMSO): δ 0.91 (6H, d, J=6 Hz), 1.18 (2H, m), 1.42 (2H, m), 1.72 (1H, m), 1.87 (1H, m), 2.42 (2H, ABq, J=16, 24 Hz), 2.90 (2H, ABq, J=12, 34 Hz), 8.00 (3H, br s), 12.34 (1H, br s).

MS (ES+) m/e: 186 ([MH-HCl]⁺, 100%).

### EXAMPLE 8

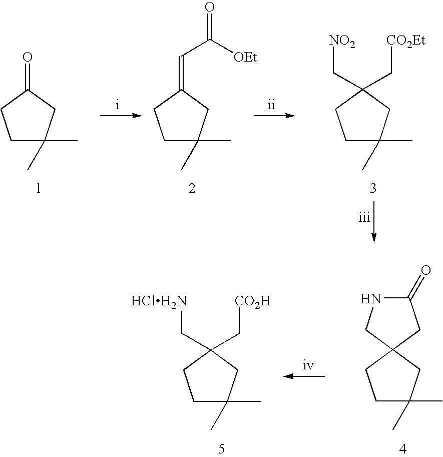

Reagents and conditions: (i) (EtO)₂POCH₂CO₂Et, NaH, THF; (ii) CH₃NO₂, nBu₄NF, THF; (iii) RaNi, H₂, MeOH; (iv) 6N HCl.

Synthesis of the Dimethylcyclopentanone 1

3,3-Dimethylcyclopentanone was prepared according to the procedure of Hiegel and Burk, J. Org. Chem., 1973;38:3637.

Synthesis of (3,3-Dimethyl-cyclopentylidene)-acetic Acid Ethyl Ester (2)

To a stirred solution of triethylphosphonoacetate (1.84 g, 7.52 mmol) in TBF (20 mL) at 0° C. was added sodium hydride (300 mg of a 60% dispersion in oil). After 30 minutes, the ketone 1 (766 mg, 6.84 mmol) in TBF (5 mL) was added. After 24 hours, the solution was diluted with a saturated solution of ammonium chloride and the two phases separated. The aqueous phase was extracted with diethyl ether (3×50 mL) and dried (MgSO₄). The combined organic phases were concentrated and flash chromatographed (25:1 hexane/ethyl acetate) to give the ester 2 as an oil, (697 mg, 56%).

¹H NMR (400 MHz, CDCl₃): δ 5.7 (1H, s), 4.1 (2H, q), 2.8 (1H, t), 2.5 (1H, t), 2.2 (1H, s), 1.55 (1H, m), 1.45 (1H, m), 1.2 (3H, t), 1.0 (3H, s), 0.98 (3H, s).

MS (m/z): 183 (MH⁺, 100%), 224 (50%).

Synthesis of (±)-(3,3-Dimethyl-1-nitromethyl-cyclopentyl)-acetic Acid Ethyl Ester (3)

Tetrabutylammonium fluoride (5.75 mL of a 1 M solution in THF, 5.75 mmol) was added to a solution of the ester 2 (697 mg, 3.83 mmol) and nitromethane (467 mg, 7.66 mmol) in THF (20 mL) and the mixture heated to 70° C. After 19 hours, nitromethane (233 mg, 1.9 mmol) and tetrabutylammonium fluoride (1.9 mL of a 1 M solution in TKF, 1.9 mmol) were added and reflux continued for 7 hours, whereupon the solution was cooled to room temperature, diluted with ethyl acetate (40 mL), and washed with 2N HCl (20 mL) then brine (20 mL). The organic phase was dried (MgSO₄) and concentrated. The crude product was flash chromatographed (9:1 hexane/ethyl acetate) to give the nitro ester 3 (380 mg, 41%) as an oil.

¹H NMR (400 MHz, CDCl₃): δ 4.62 (1H, d), 4.6 (1H, d), 4.1 (2H, q), 2.6 (1H, d), 2.58 (1H, d), 1.8 (1H, m), 1.7 (1H, m), 1.6-1.4 (4H, m), 1.2 (3H, t), 0.98 (6H, s).

MS (m/z): 244 (MH⁺, 40%), 198 (100%).

Synthesis of (±)-7,7-Dimethyl-2-aza-spiro[4.4]nonan-2-one (4)

The ester 3 (380 mg, 1.6 mmol) and Raney Nickel (1 g) were suspended in methanol (75 mL) and shaken under a hydrogen atmosphere for 24 hours. The catalyst was removed by filtration, the filtrate concentrated to give the lactam 4 (246 mg, 94%) as a white solid.

¹H NMR (400 MHz, CD₃OD): δ 3.21 (1H, d), 3.08 (1H, d), 2.24 (1H, d), 2.18 (1H, d), 1.7 (2H, m), 1.5-1.4 (4H, m), 0.98 (6H, s).

MS (m/z): 168 (MH⁺, 40%).

Synthesis of (±)-(1-Aminomethyl-3,3-dimethyl-cyclopentyl)-acetic Acid Hydrochloride (5)

The lactam (240 mg, 1.44 mmol) in 6N HCl were heated to reflux for 24 hours. The residue was concentrated under reduced pressure and triturated with ether to give the amino acid 5 as a white solid.

¹H NMR (400 MHz, CD₃OD): δ 2.98 (2H, s), 2.4 (2H, s), 1.5 (2H, m), 1.4-1.2 (4H, m), 0.84 (3H, s), 0.84 (3H, s).

MS (m/z): 186 (MH⁺, 100%), 168 (M-NH₃, 20%).

### EXAMPLE 9

Synthesis of (cis)-(3R)-(1-Aminomethyl-3-methyl-cyclopentyl)-acetic Acid Hydrochloride

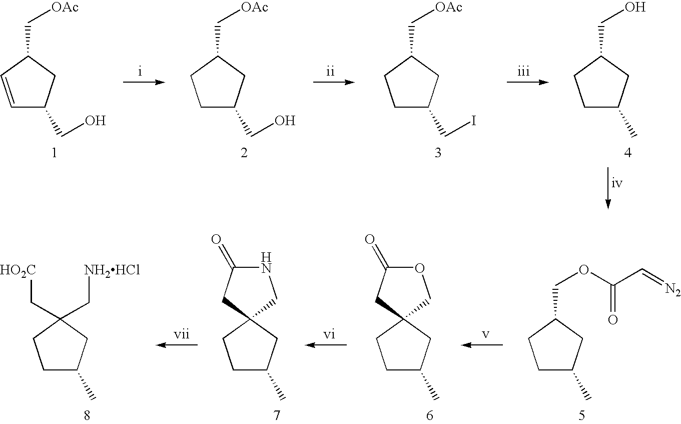

Reagents and conditions: (i) H₂, Pd/C, MeOH; (ii) I₂, Ph₃P, imidazole, CH₃CN; (iii) LAH, THF; (iv) TsNHN═CHCOCl, PhNMe₂, Et₃N; (v) Rh₂(cap)₄, CH₂Cl₂, reflux; (vi) a) BBr₃, EtOH; b) NH₃; (vii) 6N HCl, reflux.

The monoester 1 was prepared according to the procedure described in

Tetrahedron: Asymmetry 3, 1992:431.

In the first step, the ester 1 is hydrogenated using catalysts such as Raney nickel, palladium on charcoal or rhodium catalyst or other nickel or palladium containing catalyst in a solvent such as methanol, ethanol, isopropanol, ethyl acetate, acetic acid, 1,4-dioxane, chloroform or diethyl ether at a suitable temperature in the range from 20° C. to 80° C.

In the second step, the alcohol 2 is treated with triphenylphosphine, imidazole, and iodine in a solvent such as ether, tetrahydrofuran, or acetonitrile at 0° C. to room temperature to give the iodide 3.

In the third step, the iodide 3 is treated with a suitable reducing agent such as lithium aluminum hydride or lithium borohydride in a solvent such as ether or tetrahydrofuran at temperature between 0C and or reflux to give the alcohol 4. In step four, the alcohol 4 is treated with glyoxylic acid chloride (p-toluenesulfonyl)hydrazone and N,N-dimethylaniline followed by triethylamine in a solvent such as methylene chloride, chloroform, benzene, or toluene to give the diazoacetate 5.

What is claimed is:

1. A method for treating a disorder in a mammal, including a human, comprising administering to said mammal a therapeutically effective amount of a compound of formula 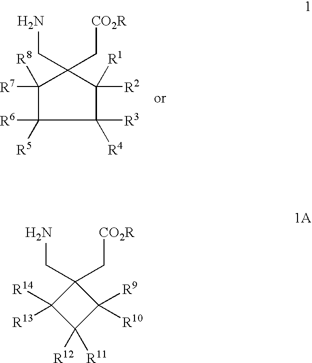
or a pharmaceutically acceptable salt thereof wherein
R is hydrogen or a lower alkyl;
R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen, and wherein
said disorder is selected from OCD, phobias, PTSD, and fibromyalgia.

2. The method according to claim 1 wherein the compound administered is (3S, 4S)-(1-Aminomethyl-3,4-dimethyl-cyclopentyl)-acetic acid.

3. The method according to claim 1 wherein said disorder is OCD, and wherein the compound administered is (3S, 4S)-(1-Aminomethyl-3,4-dimethyl-cyclopentyl)-acetic acid.

4. The method according to claim 1 wherein said disorder is PTSD, and wherein the compound administered is (3S, 4S)-(1-Aminomethyl-3,4-dimethyl-cyclopentyl)-acetic acid.

5. The method according to claim 1 wherein said disorder is a phobia, and wherein the compound administered is (3S, 4S)-(1-Aminomethyl-3,4-dimethyl-cyclopentyl)-acetic acid.

6. The method according to claim 1 wherein said phobia is selected from agoraphobia and specific phobias, and wherein the compound administered is (3S, 4S)-(1-Aminomethyl-3,4-dimethyl-cyclopentyl)-acetic acid.

7. A method for treating fibromyalgia in a mammal, including a human, comprising administering to said mammal a therapeutically effective amount of a compound of formula 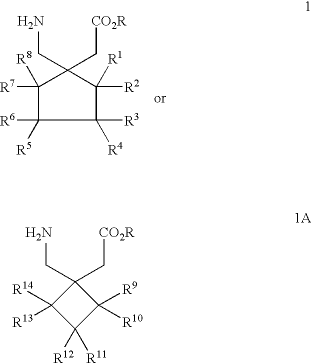
or a pharmaceutically acceptable salt thereof wherein
R is hydrogen or a lower alkyl;
R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen.

8. A method according to claim 7, wherein the compound administered is (3S, 4S)-(1-Aminomethyl-3,4-dimethyl-cyclopentyl)-acetic acid.

9. A method for treating fibromyalgia and a concomitant disorder in a mammal, including a human, comprising administering to said mammal a therapeutically effective amount of a compound of formula 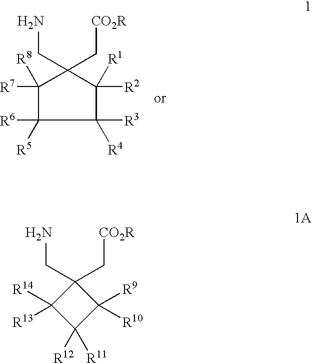
or a pharmaceutically acceptable salt thereof wherein
R is hydrogen or a lower alkyl;
R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen, and wherein
said concomitant disorder is selected from migraine headaches, temporomandibular joint dysfunction, dysautonomia, endocrine dysfunction, dizziness, cold intolerance, chemical sensitivity, sicca symptoms, cognitive dysfunction, generalized anxiety disorder, premenstrual dysphoric dysthemia, irritable bowel syndrome, functional abdominal pain, neuropathic pain, and somatoform disorders, OCD, phobias, and PTSD.

10. The method according to claim 9 wherein the compound administered is (3S, 4S)-(1-Aminomethyl-3,4-dimethyl-cyclopentyl)-acetic acid.

11. The method according to claim 9 wherein said concomitant disorder is generalized anxiety disorder, premenstrual dysphoric disorder, or a somatoform disorder, and wherein the compound administered is (3S, 4S)-(1-Aminomethyl-3,4-dimethyl-cyclopentyl)-acetic acid.

12. The method according to 9 wherein said concomitant disorder is irritable bowel syndrome, functional abdominal pain, neuropathic pain, or migraine headache, and wherein the compound administered is (3S, 4S)-(1-Aminomethyl-3,4-dimethyl-cyclopentyl)-acetic acid.

13. A method of increasing slow wave sleep in a human subject being treated with an active pharmaceutical agent that decreases slow wave sleep comprising administering to a human subject in need of such treatment:
(a) a compound of the formula 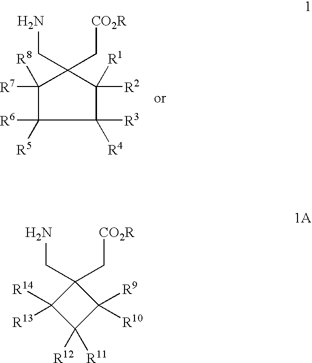
or a pharmaceutically acceptable salt thereof wherein
R is hydrogen or a lower alkyl;
R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen; and
(b) a human growth hormone or human growth hormone secretagogue, or a pharmaceutically acceptable salt thereof;
wherein the amounts of the active agents “a” and “b” are chosen so as to render the combination effective in increasing slow wave sleep.

14. The method of claim 13 wherein said compound is (3S, 4S)-(1-Aminomethyl-3,4-dimethyl-cyclopentyl)-acetic acid.

15. A method of increasing slow wave sleep in a human subject comprising administering to a human subject in need of such treatment:
(a) a compound of the formula 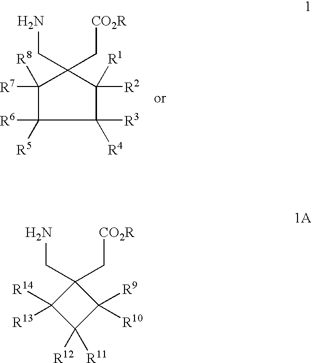
or a pharmaceutically acceptable salt thereof wherein
R is hydrogen or a lower alkyl;
R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen; and
(b) a human growth hormone or human growth hormone secretagogue, or a pharmaceutically acceptable salt thereof;
wherein the amounts of the active agents “a” and “b” are chosen so as to render the combination effective in increasing slow wave sleep.

16. The method of claim 15 wherein said compound is (3S, 4S)-(1-Aminomethyl-3,4-dimethyl-cyclopentyl)-acetic acid.
